(19) United States
(12) Patent Application Publication
(10) Pub. No.: US 2013/0310421 A1
(43) Pub. Date: Nov. 21, 2013

(54) TREATMENT OF AMYLOID BETA AMYLOIDOSIS

(71) Applicant: Temple University - Of The Commonwealth System of Higher Education, (US)

(72) Inventor: Domenico Pratico, Philadelphia, PA (US)

(73) Assignee: Temple University - Of The Commonwealth System of Higher Education, Philadelphia, PA (US)

(21) Appl. No.: 13/826,223

(22) Filed: Mar. 14, 2013

Related U.S. Application Data
(60) Provisional application No. 61/649,085, filed on May 18, 2012.

(51) Int. Cl. A61K 31/4709

(52) U.S. Cl. CPC A61K 31/4709
USPC 514/314

(57) ABSTRACT

Provided is a method for the treatment and/or prevention of an amyloid beta amyloidosis in a subject comprising the step of administering to the subject an effective amount of a 5-lipoxygenase-activating protein (FLAP) antagonist. In preferred embodiments, the FLAP antagonist is MK-591, or a pharmaceutically acceptable salt thereof. Also provided is a method for decreasing or preventing the deposition of beta amyloid in the brain of a subject comprising the step of administering to a subject an effective amount of a FLAP antagonist. In preferred embodiments, the FLAP antagonist is MK-591, or a pharmaceutically acceptable salt thereof.

### REFERENCE TO GOVERNMENT GRANT

The invention was made with government support under grant nos. AG033568 and NS071096 awarded by the National Institutes of Health. The government has certain rights in the invention.

### FIELD OF THE INVENTION

The invention relates to the field of biotechnology and neuroscience, and in particular to the treatment of amyloid beta amyloidosis.

### BACKGROUND OF THE INVENTION

The 5-lipoxygenase (5LO) enzyme catalyzes the conversion of arachidonic acid to 5-hydroxy-peroxy-eicosatetraenoic acid (5-HPETE) and subsequently to 5-hydroxy-eicosatetraenoic acid (5-HETE), which can then be metabolized into different leukotrienes (Murphy R C, Gijon M A, Biochem J. 2007, 405:379-395). 5LO is abundantly present in the central nervous system (CNS), where its activity is regulated by the presence and availability of another protein, called 5LO-activating protein or FLAP (Mandal A K et al. Proc. Natl. Acad. Sci. USA 2004, 101:6587-6592). From a biochemical point of view, FLAP and 5LO form a functional complex whose integrity is necessary for full 5LO enzymatic activity. A peculiar aspect of the FLAP/5LO pathway is the fact that its expression levels are significantly increased in the CNS with aging, and that this increase is also region-specific since it mainly manifests in the hypocampus, an area of the brain vulnerable to neurodegenerative insults (Chinnici C M et al. Neurobiol. Aging 2007, 28:1457-1462).

There remains a need for a method of treating amyloid beta amyloidosis.

### SUMMARY

Provided is a method for the treatment and/or prevention of an amyloid beta amyloidosis in a subject comprising the step of administering to the subject an effective amount of a 5-lipoxygenase-activating protein (FLAP) antagonist. In preferred embodiments, the FLAP antagonist is MK-591, or a pharmaceutically acceptable salt thereof. In some embodiments, the amount is from about 0.1 mg/day to about 10 g/day. In further embodiments, the amount is from about 1 mg/day to about 5 g/day. In yet further embodiments, the amount is from about 10 mg/day to about 1 g/day. In further embodiments, the amount is from about 100 mg/day to about 800 mg/day. In some embodiments, the amount is given once a day. In further embodiments, the amount is divided into 2, 3 or 4 daily doses. In some embodiments, the disease is Alzheimer's Disease, Cerebral amyloid angiopathy, Lewy body dementia or inclusion body myositis. In preferred embodiments, the disease is Alzheimer's Disease. In further preferred embodiments, the subject is human.

Provided is a method for decreasing or preventing the deposition of beta amyloid in the brain of a subject comprising the step of administering to a subject an effective amount of a FLAP antagonist. In preferred embodiments, the FLAP antagonist is MK-591, or a pharmaceutically acceptable salt thereof. In some embodiments, the amount is from about 0.1 mg/day to about 10 g/day. In further embodiments, the amount is from about 1 mg/day to about 5 g/day. In yet further embodiments, the amount is from about 10 mg/day to about 1 g/day. In further embodiments, the amount is from about 100 mg/day to about 800 mg/day. In some embodiments, the amount is given once a day. In further embodiments, the amount is divided into 2, 3 or 4 daily doses. In some embodiments, the disease is Alzheimer's Disease, Cerebral amyloid angiopathy, Lewy body dementia or inclusion body myositis. In preferred embodiments, the disease is Alzheimer's Disease. In further preferred embodiments, the subject is human.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

### DESCRIPTION OF THE FIGURES

FIGS. 1A-D show that pharmacologic blockade of FLAP decreases brain Aβ peptide levels and deposition. FIG. 1A shows RIPA-soluble (RIPA) Aβ1-40 levels in cortex and hippocampus of Tg2576 receiving MK-591 or placebo for 8 months as measured by sandwich ELISA. (n=9 for control, and n=11 for MK-591; *p<0.04). FIG. 1B shows formic acid extractable (FA) Aβ1-40 levels in cortex and hippocampus of Tg2576 receiving MK-591 or placebo for 8 months as measured by sandwich ELISA. (n=9 for control, and n=11 for MK-591; *p<0.04). FIG. 1C shows RIPA Aβ1-42 levels in cortex and hippocampus of Tg2576 receiving MK-591 or placebo for 8 months as measured by sandwich ELISA. (n=9 for control, and n=11 for MK-591; *p<0.04). FIG. 1D shows FA Aβ1-42 levels in cortex and hippocampus of Tg2576 receiving MK-591 or placebo for 8 months as measured by sandwich ELISA. (n=9 for control, and n=11 for MK-591; *p<0.04). FIG. 1E illustrates representative sections of brains from Tg2576 mice receiving MK-591 or placebo (control) for 8 months immunostained with 4G8 antibody. FIG. 1F illustrates the quantification of the area occupied by Aβ immunoreactivity in brain of Tg2576 mice receiving MK-591 or placebo for 8 months (*p=0.03).

FIGS. 2A-E show that pharmacologic blockade of FLAP alters APP metabolism via the γ-secretase pathway. FIG. 2A shows representative western blots of APP, ADAM-10, BACE-1, sAPPα, sAPPIβ, CTFs, PS1, Nicastrin, APH-1 and Pen-2 in brain homogenates of Tg2576 mice receiving MK-591 or placebo for 8 months. FIG. 2B shows the densitometric analyses of some of the immunoreactivities to the antibodies shown in FIG. 2A (*p<0.04). FIG. 2C shows the relative mRNA levels for BACE-1, PS1, Nicastrin, APH-1 and Pen-2 in brain homogenates of Tg2576 mice receiving MK-591 or placebo for 8 months, as determined by real-time quantitative RT-PCR amplification (*p<0.02). FIG. 2D shows a representative western blot for total CREB and its phosphorylated form at Ser133, and Sp1 in brain homogenates of Tg2576 mice receiving MK-591 or placebo. FIG. 2E shows densitometric analyses of the immunoreactivities to the antibodies shown in FIG. 2D (*p<0.03).

FIGS. 3A-D show that pharmacologic blockade of FLAP reduces neuroinflammation. FIG. 3A and FIG. 3C show that representative brain sections from Tg2576 receiving MK-591 or vehicle (control) immunostained for CD45 and GFAP (×20 magnification). FIG. 3B and FIG. 3D show quantitative analyses of the immunoreactivity of antibodies for CD45 and GFAP in the same animals (*p=0.01).

FIGS. 4A-C show the in vitro effect of MK-591 on Aβ formation and APP metabolism. N2A-APPswe cells were incubated with increasing concentrations of MK-591 or vehicle for 24 hours, and conditioned media and cell lysates were collected. FIG. 4A shows Aβ1-40 levels in the cell supernatant assayed by sandwich ELISA (*p<0.01). FIG. 4B shows representative western blots of APP, ADAM-10, BACE-1, PS1, nicastrin, APH-1, and Pen-2 in the lysates of MK-591-treated or vehicle-treated cells. FIG. 4C shows densitometric analyses of the immunoreactivities to the antibodies shown in FIG. 4B (*p<0.01).

FIGS. 5A-D show the in vitro effect of MK-591 on CREB and Notch. N2A-APPswe cells were incubated with increasing concentrations of MK-591 or vehicle for 24 hours, and cell lysates collected for immunoassays. FIG. 5A shows representative western blots of total CREB, its phosphorylated form at Ser133, and Sp1 levels. FIG. 5B shows densitometric analyses of the immunoreactivities of the antibodies to CREB, p-CREB and Sp1 (*p<0.01). FIG. 5C shows representative western blots of NICD in the lysates of MK-591 or vehicle groups. FIG. 5D shows densitometric analyses of the immunoreactivities of the antibodies for NICD (*p<0.01).

FIGS. 6A-D show that genetic absence of FLAP and its pharmacological inhibition with MK-591 ameliorates behavioral deficits in 3xTg mice. FIG. 6A shows the total number of arm entries for WT, FLAPKO, 3xTg, 3xTg-FLAPKO and 3xTg-MK-591 mice at 6-8 and 12-14 months of age. FIG. 6B shows the percentage of alternations between the same group of mice (*p<0.001). FIG. 6C shows the contextual fear memory response in WT, FLAPKO, 3xTg, 3xTg-FLAPKO and 3xTg-MK-591 mice. FIG. 6D shows cued recall fear memory response in the same groups of mice (6-8 months: n=8 for WT, n=7 for FLAPKO, n=12 for 3xTg, n=12 for 3xTg-FLAPKO, n=10 for 3xTg-MK-591) (*p<0.001). Values represent mean +/− standard error of the mean.

FIGS. 7A-G show that FLAP reduction modulates Aβ peptide deposition levels in brains of 3xTg mice. FIG. 7A shows a radioimmunoprecipitation assay of RIPA-soluble extractable Aβ1-40 levels in cortex of 3xTg, 3xTg-FLAPKO and 3xTg-MK-591 mice at 14 months of age (n=9 for 3xTg and n=11 for 3xTg-FLAPKO) (*p<0.001). FIG. 7B shows formic acid (FA) extractable Aβ1-40 levels in cortex of 3xTg, 3xTg-FLAPKO and 3xTg-MK-591 mice at 14 months of age (n=9 for 3xTg and n=11 for 3xTg-FLAPKO) (*p<0.001). FIG. 7C shows a radioimmunoprecipitation assay of RIPA-soluble extractable Aβ1-42 levels in the cortex of 3xTg, 3xTg-FLAPKO and exTg-MK-591 mice at 14 months of age (n=9 for 3xTg and n=11 for 3xTg-FLAPKO) (*p<0.001). FIG. 7D shows formic acid (FA) extractable Aβ1-42 levels in the cortex of 3xTg, 3xTg-FLAPKO and 3xTg-MK-591 mice at 14 months of age (n=9 for 3xTg and n=11 for 3xTg-FLAPKO) (*p<0.001). FIG. 7E shows the quantification of the area occupied with Aβ immunoreactivity in the brains of the same group of mice (*p<0.001). FIG. 7F shows densitometric analyses of the immunoreactivities of PS-1, Nicastrin, APH-1 and Pen-2 to their respective antibodies, as measured on a western blot (not shown) (*p<0.01) (n=4 for 3xTg; n=4 for 3xTg-FLAPKO). FIG. 7G shows densitometric analyses of the immunoreactivities of PS-1, Nicastrin, APH-1 and Pen-2 to their respective antibodies, as measured on a western blot (not shown) (*p<0.01) (n=4 for 3xTg; n=4 for 3xTg-MK-591). Values represent mean+/− standard error of the mean.

FIGS. 8A-D show that FLAP modulates tau phosphorylation and metabolism in the brains of 3xTg mice at 14 months of age. FIG. 8A shows densitometric analyses of the immunoreactivities of PHF-13 and PHF-1 to their respective antibodies, as measured on a western blot for 3xTg and 3xTg-FLAPKO mice (not shown) (*p<0.01). FIG. 8B shows shows densitometric analyses of the immunoreactivities of PHF-13 and PHF-1 to their respective antibodies, as measured on a western blot for 3xTg and 3xTg-MK-591 mice (not shown) (*p<0.01). FIG. 8C shows densitometric analyses of the immunoreactivities of insoluble Tau to its antibodies, as measured on a western blot for 3xTg and 3xTg-FLAPKO mice (not shown) (*p<0.01). FIG. 8D shows densitometric analyses of the immunoreactivities of insoluble Tau to its antibodies, as measured on a western blot for 3xTg and 3xTg-MK-591 mice (not shown) (*p<0.01).

FIGS. 9A-B show that Tau hypophosphorylation in the genetic absence of FLAP and during pharmacological inhibition of FLAP by MK-591 is due to changes in specific tau kinases. FIG. 9A shows densitometric analyses of the immunoreactivities of Cdk5, p35 and p25 to their respective antibodies, as measured on a western blot (not shown) (n=4 for 3xTg; n=4 for 3xTg-FLAPKO) (*p<0.001). FIG. 9B shows densitometric analyses of the immunoreactivities of Cdk5, p35 and p25 to their respective antibodies, as measured on a western blot (not shown) (n=4 for 3xTg; n=4 for 3xTg-MK-591) (*p<0.001).

FIGS. 10A-D show that the genetic absence of FLAP and pharmacological inhibition of FLAP by MK-591 ameliorates synaptic biomarkers and decreases neuroinflammation in 3xTg mice. FIG. 10A shows densitometric analyses of the immunoreactivities if synaptophysin, MAP-2 and PSD95 to their respective antibodies, as measured on a western blot (not shown) (n=4 for 3xTg; n=4 for 3xTg-FLAPKO) (*p<0.001). FIG. 10B shows densitometric analyses of the immunoreactivities if synaptophysin, MAP-2 and PSD95 to their respective antibodies, as measured on a western blot (not shown) (n=4 for 3xTg; n=4 for 3xTg-MK-591) (*p<0.001). FIG. 10C shows densitometric analyses of the immunoreactivities if CD45 and GFAP to their respective antibodies, as measured on a western blot (not shown) (n=4 for 3xTg; n=4 for 3xTg-FLAPKO) (*p<0.001). FIG. 10D shows densitometric analyses of the immunoreactivities if CD45 and GFAP to their respective antibodies, as measured on a western blot (not shown) (n=4 for 3xTg; n=4 for 3xTg-MK-591) (*p<0.001).

FIG. 11A-B illustrates that the genetic absence of FLAP and pharmacologic inhibition of FLAP by MK-591 rescues synaptic dysfunction in 3xTg mice. FIG. 11A illustrates long-term potentiation (LTP) magnitudes expressed as the percentages of baseline for 0-10 minutes post-tetanus [274.6%+/−8.5% for WT (n=23 slices); 159.9%+/−13.8% (n=19 slices) for 3xTg; 269.7%+/−10.3% (n=21 slices) for 3xTg/FLAPKO; 272.5%+/−13.2% (n=10 slices) for 3xTg-MK-591)]. FIG. 11B shows, for the same group of mice, LTP magnitudes expressed as the percentages of baseline for 170-180 minutes post-tetanus (202.6%+/−6.5% for WT; 121.0%+/−3.4% for 3xTg; 197.1%+/−11.5% for 3xTg-FLAPKO; 156.9%+/−6.9%; 202.5%+/−9.4% for 3xTg-MK-591) (*p<0.0001). Values represent mean+/− standard error of the mean.

### DEFINITIONS

### List of Abbreviations

AD: Alzheimer's disease
5LO: 5-lipoxygenase
Tg2576 mice: transgenic mice over-expressing human Swedish mutant of APP
APP: amyloid beta precursor protein
Aβ: amyloid beta peptide
sAPPα: secreted APP alpha
sAPPβ: secreted APP beta
CTFs: C-terminal fragments
FLAP: Five-lipoxygenase activating protein
3xTg: mice harboring a mutant amyloid precursor protein (APP; KM670/671NL), a human mutant PS1 (M146V) knockin, and tau (P301L) transgenes
3xTg-FLAPKO: 3xTg mice genetically deficient for FLAP
3xTg-MK-591: 3xTg mice treated with MK-591
WT: wild type
LTP: long-term potentiation

As used in the specification and the appended claims, the singular forms “a,” “an” and “the” include plural referents unless the context clearly dictates otherwise.

The term “about” will be understood by persons of ordinary skill in the art and will vary to some extent depending on the context in which it is used. As used herein, “about” is meant to encompass variations of ±20% or ±10%, more preferably ±5%, even more preferably ±1%, and still more preferably ±0.1%.

As used herein, the terms “treat” and “treatment” are used interchangeably and are meant to indicate a postponement of development of a disorder and/or a reduction in the severity of symptoms that will or are expected to develop. The terms further include ameliorating existing symptoms, preventing additional symptoms, and ameliorating or preventing the underlying metabolic causes of symptoms.

The expression “effective amount” or “therapeutically effective amount” refers to the amount of a compound that inhibits or prevents the deposition of beta amyloid in a tissue of a subject, particularly in the brain.

As used herein, the term “subject” or “patient” refers to any animal (e.g., a mammal), including, but not limited to humans, non-human primates, rodents, and the like. Typically, the terms “subject” and “patient” are used interchangeably herein in reference to a human subject.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

### DETAILED DESCRIPTION

The presence of amyloid plaques in hippocampus, neocortex, and amygdale is another major pathological hallmark of Alzheimer's disease. The principal component of amyloid plaques is the amyloid beta peptide. Amyloid beta peptide (Aβ) is a peptide comprising 39 to 43 amino acids, processed from the amyloid precursor protein (APP), which is a transmembrane glycoprotein. Amyloid beta peptide is generated by the successive cleavage action of beta and gamma secretases. The cleavage processing of the APP can generate various isoforms of amyloid beta, ranging from 39 to 43 amino acid residues in length, e.g., amyloid beta 40 and amyloid beta 42. The latter is the more fibrillogenic and is associated with diseases genesis.

Provided are methods for the treatment and/or prevention of an amyloid beta amyloidosis in a subject comprising the step of administering to the subject in need of such treatment or prevention an effective amount of a 5-lipoxygenase-activating protein (FLAP) antagonist. The FLAP antagonists inhibit beta amyloid deposition. The data presented in the examples herein demonstrates that pharmacological blockade of FLAP with MK-591 significantly reduces brain Aβ formation and deposition in the Tg2576 mouse model of AD, establishing that therapeutic antagonism of FLAP is a novel treatment for modulating amyloidogenesis.

Accordingly, the compositions and methods of the invention are useful for inhibiting amyloidosis in disorders in which beta amyloid deposition occurs. The methods of the invention can be used therapeutically to treat beta amyloidosis or can be used prophylactically in a subject susceptible to beta amyloidosis.

As demonstrated herein, FLAP antagonist treatment reduces brain Aβ peptide levels and its deposition, and FLAP inhibition influences the metabolism of APP in part through a reduction in the steady state levels of three of the four components of the γ-secretase complex, PS1, Pen-2 and APH-1. As demonstrated herein, treatment with FLAP antagonist modulates neuroinflammation, and ameliorates cognition as demonstrated through several behavioral tests. As demonstrated herein, treatment with FLAP antagonist reduces tau phosphorylation, and ameliorates synaptic integrity.

The term “FLAP antagonist,” as used herein, means an antagonist or inhibitor of 5-lipoxygenase-activating protein (FLAP). In some embodiments, the FLAP antagonist is a small molecule drug. In preferred embodiments, the FLAP antagonist is MK-591. In further embodiments, the FLAP antagonist is an antibody or a FLAP-binding molecule.

The term “MK-591,” as used herein, means 3-[3-tert-butylsulfanyl-1-[(4-chlorophenyl)methyl]-5-(quinolin-2-ylmethoxy)indol-2-yl]-2,2,-dimethylpropanoic acid. The CAS Number for MK-591 is 136668-42-3. The structure of MK-591 is shown below:

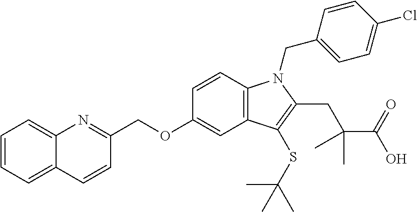 [SMILES: CC(C)(C)Sc1c(CC(C)(C)C(=O)O)n(Cc2ccc(Cl)cc2)c2ccc(OCc3ccc4ccccc4n3)cc12]

By “amyloid beta amyloidosis” is meant an amyloidosis associated with the deposition of a beta amyloid peptide. “Amyloidosis” is intended to have its normal meaning in medicine, i.e., a condition charcaterized by the abnormal deposition of amyloid proteins in organs or tissues.

The methods of the invention involve administering to a subject a therapeutic compound which inhibits beta amyloid deposition “Inhibition of beta amyloid deposition” is intended to encompass prevention of beta amyloid formation, inhibition of further beta amyloid deposition in a subject with ongoing amyloidosis and reduction of beta amyloid deposits in a subject with ongoing beta amyloidosis.

Amyloid beta amyloidosis is a neurodegenerative disease that involves the formation of amyloid beta amyloidosis plaques. Amyloid beta amyloidosis involves production of amyloid beta by truncation or cleavage processing of the amyloid precursor protein (APP). Thus, in some aspects, an amyloid beta amyloidosis treated according to the present invention may be Alzheimer's disease (AD), Cerebral amyloid angiopathy, Inclusion body myositis, or variants of Lewy body dementia. For instance, in certain aspects the invention provides methods and compositions for delaying the onset or progression of AD. However, in certain other aspects, methods for treating, delaying the onset or delaying the progression of a non-Alzheimer amyloid beta amyloidosis are provided.

A “subject” may be any animal, though in a preferred embodiment, the subject is a human. In certain embodiments, the methods of the invention involve delaying or preventing the progression of an amyloid beta amyloidosis in a subject. Thus, in certain aspects, a subject may be a subject that has been diagnosed with a amyloid beta amyloidosis. For example, a subject may be defined as a person who has been diagnosed with AD or has clinical and/or pathological signs of AD. In still other embodiments, there is provided a method for preventing or delaying the onset of amyloid beta amyloidosis. Thus, the skilled artisan will recognize in some cases subjects are defined as not yet having an amyloid beta amyloidosis. For example, in some aspects, a subject may be at risk for developing an amyloid beta amyloidosis. An at risk subject may, for instance, have a genetic predisposition to amyloid beta amyloidosis (e.g., as ascertained by family history or a genetic mutation). In still further cases, an at risk subject may lack clinical disease but comprise risk factors for disease such as declining cognitive (e.g., mild cognitive impairment (MCI)) or memory function or elevated levels of a marker protein (e.g., amyloid beta) in the serum or CNS or increased or advancing age.

### FLAP Antagonists

FLAP antagonists may be identified based on their binding to FLAP. Several inhibitors of FLAP have been described (Gillard et al., Can. J. Physiol. Pharmacol., 67, 456-464, 1989; Evans et al., Molecular Pharmacol., 40, 22-27, 1991; Brideau et al., Can. J. Physiol. Pharmacol., 70(6):799-807, 1992; Musser et al., J. Med. Chem., 35, 2501-2524, 1992; Steinhilber, Curr. Med. Chem. 6(1):71-85, 1999; Riendeau, Bioorg Med Chem. Lett., 15(14):3352-5, 2005; Flamand, et al., Mol. Pharmacol. 62(2):250-6, 2002; Folco et al., Am. J. Respir. Crit. Care Med. 161(2 Pt 2):S112-6, 2000; Hakonarson, JAMA, 293(18):2245-56, 2005).

FLAP antagonists include, among others, indole derivatives and quinoline derivatives. Indole derivatives with FLAP inhibitory activity include, by way of example and not limitation, 3-[3-butylsulfanyl-1-[(4-chlorophenyl)methyl]-5-propan-2-yl-indol-2-yl]-2-, 2-dimethyl-propanoic acid (i.e., MK-866) and 3-[1-[4-chlorophenyl)methyl]-5-(quinolin-2-ylmethoxy)-3-tert-butylsulfan-yl-indol-2-yl]-2,2-dimethyl-propanoic acid (i.e., MK-591 or quiflapon). Quinoline derivatives include, by way of example and not limitation, (2R)-2-cyclopentyl-2-[4-(quinolin-2-ylmethoxy)phenyl]acetic acid (i.e., BAY-X1005 or veliflapon).

### Administration

The compounds used in the methods of the present invention may be administered by any route, including oral and parenteral administration. Parenteral administration includes, for example, intravenous, intramuscular, intraarterial, intraperitoneal, intranasal, rectal, intravesical, intradermal, topical or subcutaneous administration. Also contemplated within the scope of the invention is the instillation of drug in the body of the patient in a controlled formulation, with systemic or local release of the drug to occur at a later time. For example, the drug may be localized in a depot for controlled release to the circulation, or for release to a local site of tumor growth.

The specific dose of compound to obtain therapeutic benefit for treatment of a proliferative disorder will, of course, be determined by the particular circumstances of the individual patient including, the size, weight, age and sex of the patient, the stage of the disease, the aggressiveness of the disease, and the route of administration of the compound.

The daily dose of the compound may be given in a single dose, or may be divided, for example into two, three, or four doses, equal or unequal, but preferably equal, that comprise the daily dose. When given intravenously, such doses may be given as a bolus dose injected over, for example, about 1 to about 4 hours.

The compounds used in the methods of the present invention may be administered in the form of a pharmaceutical composition, in combination with a pharmaceutically acceptable carrier. The active ingredient in such formulations may comprise from 0.1 to 99.99 weight percent. By “pharmaceutically acceptable carrier” is meant any carrier, diluent or excipient which is compatible with the other ingredients of the formulation and not deleterious to the recipient.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The pharmaceutical composition is preferably in unit dosage form. In such form the preparation is divided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The practice of the invention is illustrated by the following non-limiting examples. The invention should not be construed to be limited solely to the compositions and methods described herein, but should be construed to include other compositions and methods as well. One of skill in the art will know that other compositions and methods are available to perform the procedures described herein.

### EXAMPLES

### Example 1

### FLAP Blockade Reduces Brain Aβ Peptide Levels and its Deposition

### A. Procedures

### Tg2576 Mice and Treatments

The Tg2576 transgenic mice expressing human APP with the Swedish mutation (K670N/M671L) used in these studies were previously described (Pratico D et al. J Neurosci 2001, 21(12):4183-4187). The mice were genotyped by polymerase chain reaction (PCR) analysis using tail DNA and kept in a pathogen-free environment, one 12-hour light/dark cycle and had access to food and water ad libitum. All the experiments presented herein were performed with female mice. Starting at 7 months of age, mice were randomized to receive MK-591 (40 mg/kg weight) (n=11) or vehicle (n=9) in their chow diet for 8 months until they were 15 months old. Considering that each mouse eats on average 5 g/day of chow diet and the diet is formulated for 320 mg MK-591 pwer kg diet (Harlan Teklad, Wis., USA), the final dose of the active drug was approximately 40 mg/kg weight/day. During the study, mice in both groups gained weight regularly, and no significant difference in weight was detected between the two groups. No macroscopic effect on the overall general health was observed in the animals receiving the active treatment. Post-mortem examination showed no sign of macroscopic pathology in any of the organs considered (i.e., spleen, liver, thymus, ileum).

After sacrifice, animals were perfused with ice-cold 0.9% Phosphate Buffered Saline (PBS), their brain was removed and dissected in two hemihalves by midsagittal dissection. One was immediately stored at −80° C. for biochemistry assays or total RNA extraction, and the other immediately immersed in 4% paraformaldehyde in 0.1 M PBS (pH 7.6) overnight for immunohistochemistry studies.

### Immunohistochemistry

Immunostaining was performed as reported by Yang H et al. Biol Psych 2010, 68(10):922-929, Chu J et al. Mol. Neurodegen. 2012, 7:1. Serial 6-μm-thick coronal sections were mounted on 3-aminopropyl triethoxysilane (APES)-coated slides. Every eighth section from the habenular to the posterior commissure (8-10 sections per animal) was examined using unbiased stereological principles. The sections for Aβ were deparaffinized, hydrated, pretreated with formic acid (88%) and subsequently with 3% H₂O₂ in methanol. The sections for GAFP and CD45 were deparaffinized, hydrated and treated with 3% H₂O₂ in methanol and subsequently antigen retrieved with citrate (10 mM).

Sections were blocked in 2% fetal bovine serum before incubation with primary antibodies (4G8 for Aβ, anti-GFAP, and antiCD45) overnight at 4° C. Subsequently, sections were incubated with biotinylated anti-mouse IgG (Vector Lab., Burlingame, Calif., USA) and then developed by using the avidin-biotin complex method (Vector Lab) with 3,3′-diaminobenzidine (DAB) as a chromogen. Light microscopic images were used to calculate the are occupied by Aβ-immunoreactivity using the software Image-Pro Plus for Windows version 5.0 (Media Cybernetics). The threshold optical density that discriminated staining from background was determined and kept constant for all quantifications. The area occupied by Aβ-immunoreactivity was measured by the software and divided by the total area of interest to obtain the percentage area of Aβ-immunoreactivity.

### Biochemical Analyses

Mouse brain homogenates were sequentially extracted first in RIPA (25 mM Tris-HCl pH 7.6, 150 mM NaCl, 1% NP-40, 1% sodium deoxycholate, 0.1% SDS) for the Aβ soluble fractions and then in formic acid (FA) for the Aβ insoluble fractions as previously described (Firuzi O et al. FASEB J2008, 22:1169-1178; Yang H et al. Biol Psych 2010, 68(10):922-929, Chu J et al. Mol. Neurodegen. 2012, 7:1). Aβ1-40 and Aβ1-42 levels were assayed by a sensitive sandwich ELISA kits (WAKO Chem.). Analyses were always performed in duplicate and in a coded fashion.

### Western Blot Analyses

RIPA extracts from brain homogenates were used for western blot analyses. Samples were electrophoresed on 10% Bis-Tris gels or 3-8% Tris-acetate gel (Bio-Rad, Richmond, Calif., USA), according to the molecular weight of the target molecule, transferred onto nitrocellulose membranes (Bio-Rad), and then incubated with antibodies as follows: anti-APP N-terminal raised against amino acids 66-81 for total APP (22C11, Chemicon International, USA), anti-BACE-1 (IBL America, USA), anti-ADAM-10 (Chemicon, USA), anti-sAPPa (2B3, IBL America), anti-sAPPIβ (6A1, IBL America), anti-CTFs (EMD Biosci. Inc.), anti-PS1 (Sigma, USA), anti-nicastrin (Cell Signaling, USA), anti-APH-1 (Millipore, USA), anti-Pen-2 (Invitrogen, USA), anti-NICD (Cell Signaling, USA), anti-IDE N-terminal (EMD Biosci.), anti-neprilysin (Santa Cruz Biotech), anti-apoE (Santa Cruz Biotech), anti-CREB (Cell Signaling) and anti-p-CREB (Cell Signaling), anti-Sp1 (Santa Cruz Biotech), anti-β actin (Santa Cruz Biotech).

After three washings with T-TBS, membranes were incubated with IRDye 800CW or IRDye 680CW-labeled secondary antibodies (LI-COR Bioscience, Nebr., USA) at 22° C. for 1 h. Signals were developed with Odyssey Infrared Imaging Systems (LI-COR Bioscience). Beta-actin was always used as internal loading control.

### Real-Time Quantitative RT-PCR Amplification

RNA was extracted and purified using the RNeasy mini-kit (Qiagen), as previously described (Chu J et al. Ann Neurol 2011, 69:34-46). Briefly, 1 μg of total RNA was used to synthesize cDNA in a 20 μl reaction using the Rt² First Strand Kit for reverse transcriptase-PCR (Super Array Bioscience, USA). Mouse BACE-1, PS1, Nicastrin, APH-1 and Pen-2 genes were amplified by using the corresponding primers designed and synthesized by Super Array Bioscience. Beta-actin was always used as an internal control gene to normalize for the amount of RNA. Real-time PCR was performed in an Eppendorf® ep realplex thermal cycler (Eppendorf, N.Y., USA). 2 μl of cDNA was added to 25 μl of SyBR Green PCR Master Mix (Applied Biosystems, CA, USA). Each sample was run in duplicate, and analysis of relative gene expression was done by using the 2^{-ΔΔCt} method (Kenneth J L et al. Method 2002, 25:402-408). Briefly, the relative change in gene expression was calculated by subtracting the threshold cycle (ΔCt) of the target genes from the internal control gene (β-Actin). Based on the fact that the amount of cDNA doubles in each PCR cycle (assuming a PCR efficiency of 100%), the final fold-change in gene expression was calculated by using the following formula: relative change=2^{-ΔΔCt} (Kenneth J L et al. Method 2002, 25:402-408).

### Cell Cultures

N2A (neuro-2 A neuroblastoma) cells stably expressing human APP carrying the K670 N, M671 L Swedish mutation (APP swe) were grown in Dulbecco's modified Eagle medium supplemented with 10% fetal bovine serum, 100 U/ml penicillin, 100 μg/ml streptomycin (Cellgro, Herdon, Va., USA), and 400 μg/ml G418 (Invitrogen, Carlsbad, Calif.), at 37° C. in the presence of 5% CO2 as previously described (Chu J et al. Ann Neurol 2011, 69:34-46).

For each experiment, equal numbers of cells were plated in six-well plates, 24 hours later media were removed and fresh media containing either MK-591 (1, 10, 25 μM) or vehicle were added. After a 24-hour incubation, supernatants were collected for Aβ assays, and cell pellets harvested in lytic buffer for immunoblot analyses as described in the previous paragraphs.

For transfection studies, N2A-APPswe cells were transfected with 1 μg Myc-tagged mAE-Notch-1 complementary DNA overnight (a generous gift from Dr. L. D'Adamio, Albert Einstein Medical College, NY) by using Lipofectamine 2000 (Invitrogen). The media were removed and fresh media containing MK-591, L685, 458 or vehicle was added. After a 24-hour incubation, cell lysates were collected and Notch intracellular domain (NICD) expression levels assayed by western blot analysis.

### Data Analysis

Data analyses were performed using SigmaStat for Windows version 3.00. Statistical comparisons were performed by Unpaired Student's t-test or the Mann-Whitney rank sum test when a normal distribution could not be assumed. Values represent mean±S.E.M. Significance was set at p<0.05.

### B. Results

In vivo studies

### FLAP Blockade Reduces Brain Aβ Peptide Levels and Deposition

Starting at seven months of age Tg2576 mice were randomized to receive MK-591 (320 mg/kg diet) or vehicle in their chow diet for 8 months before being sacrificed. Considering that each mouse eats on average 5 g/day of chow diet, the final dose of the active drug was approximately 40 mg/kg weight/day. By the end of the study, body weight, total plasma cholesterol, triglycerides and blood cell counts were not different between the two groups (not shown).

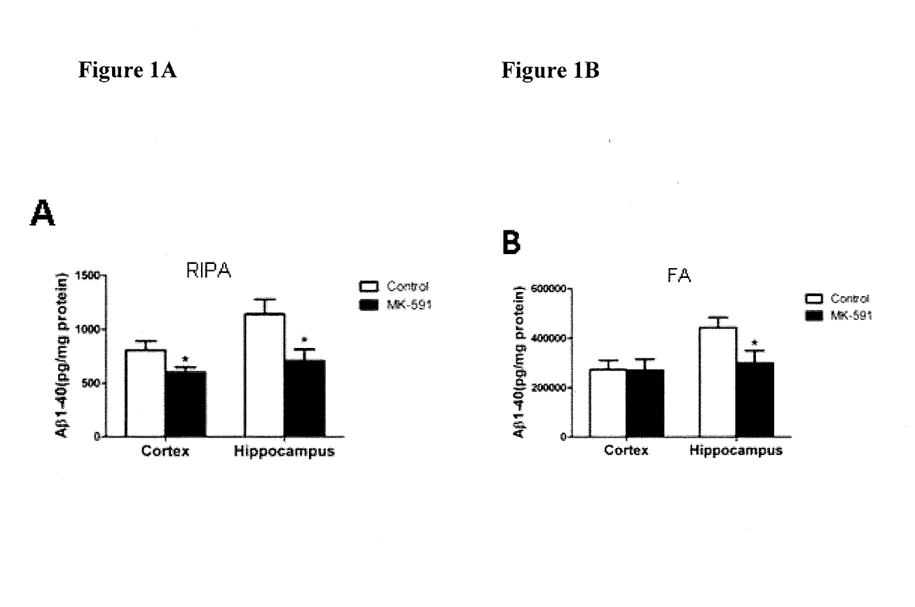

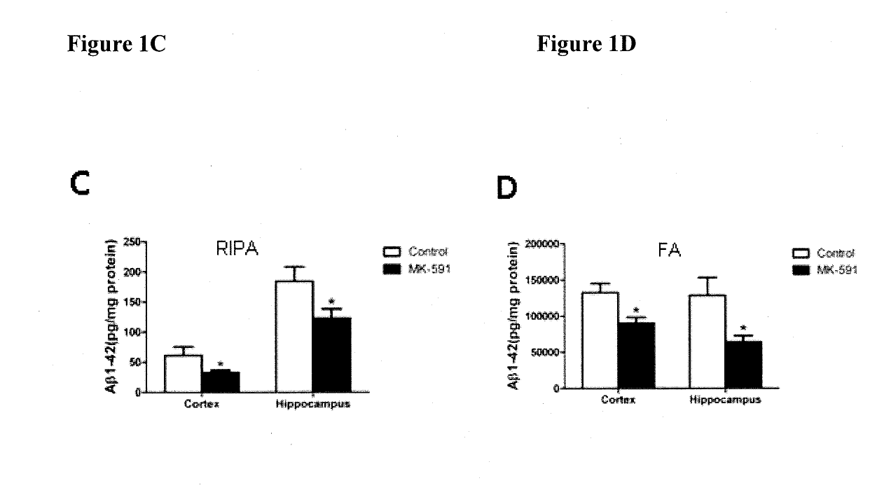

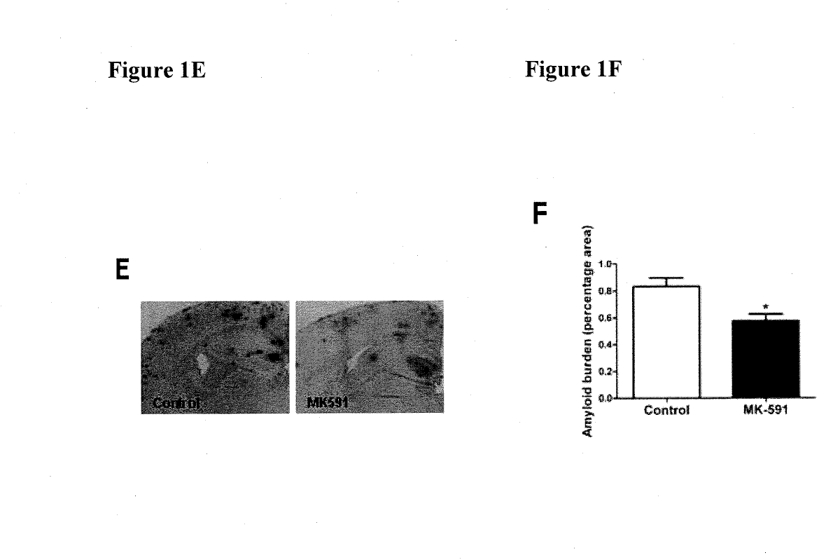

As expected for their age, 15 month-old Tg2576 mice on placebo showed elevated levels of both soluble (RIPA extractable) and insoluble (Formic acid extractable) Aβ1-40 and Aβ1-42 in their cerebral cortex as well as hippocampus, which were significantly reduced in mice receiving MK-591 (FIG. 1A-D).

Amyloid deposists were widely distributed in the cerebral cortex and hippocampus of Tg2576 mice at 15 months of age, as previously reported (Kawarabayashi T et al. J. Neurosci. 2001, 21(2):372-381). To determine the effect of chronic MK-591 administration on brain amyloid deposition, the areas occupied by 4G8-immunopositive reactions were analyzed. Comparison of the Aβ-immuno positive areas between placebo and MK-591-treated group revealed a statistically significant reduction of the amyloid burden in the treated mice (FIG. 1E, F).

### FLAP Blockade Influences Brain APP Metabolism

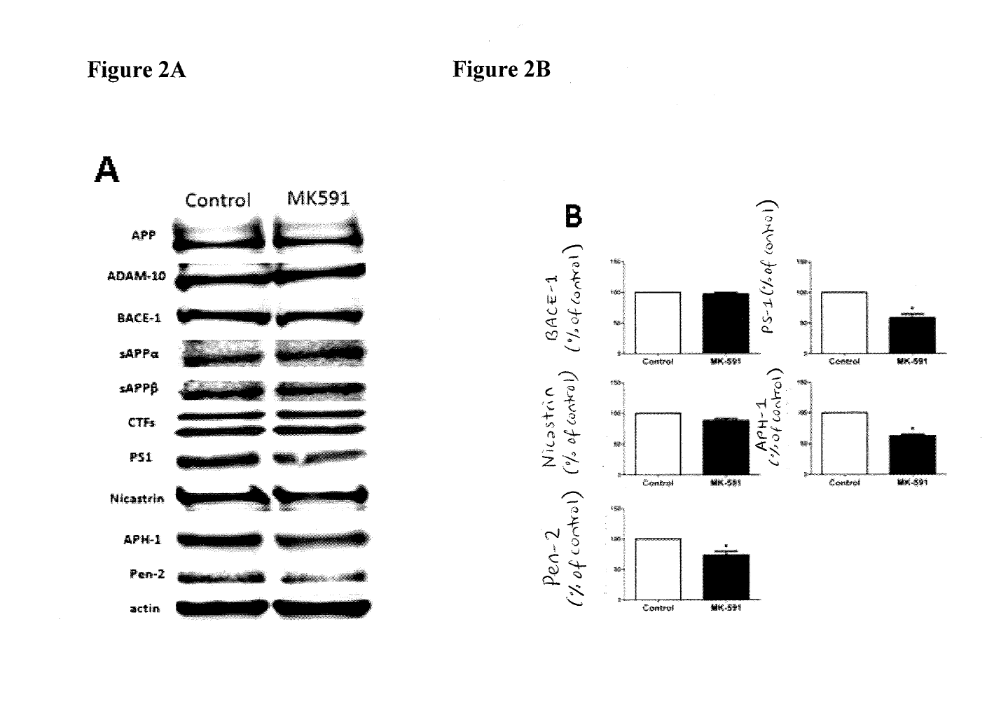

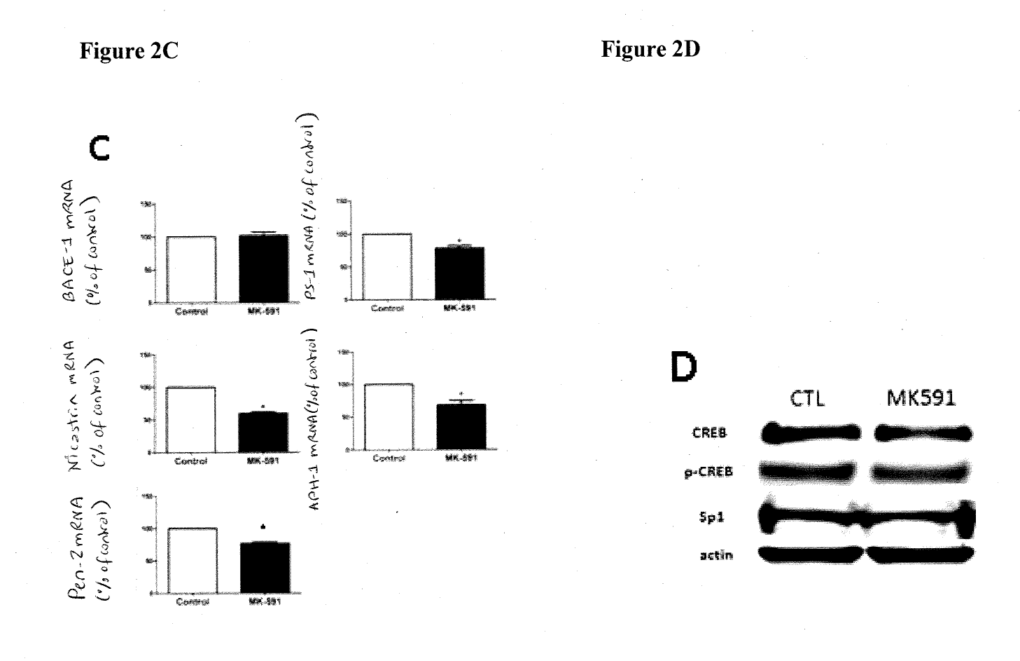

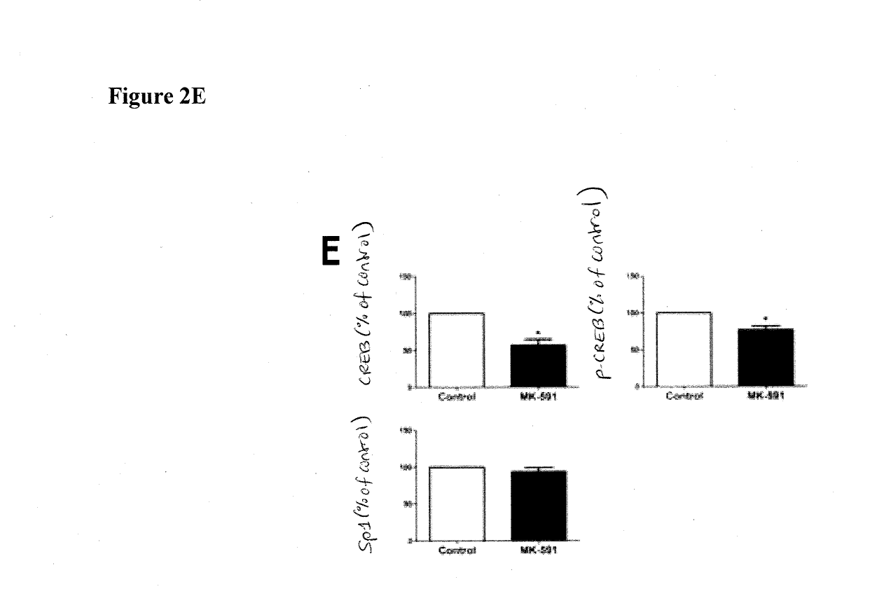

Since Aβ is the final product of the proteolytic processing of its own precursor, the amyloid β precursor protein (APP), next we investigated whether this pharmacologic treatment was associated with an alteration of the expression levels of this protein. As shown in FIG. 2A, we found that there was no difference in total APP levels between the two groups of mice: control and treated with MK-591. To assess the effect of MK-591 on APP processing we investigated the steady state levels of main enzyme proteases involved: α-secretase (ADAM-10), β-secretase (BACE-1), sAPPα, sAPPIβ, C-terminal fragments (CTFs) by western blot in control mice and in mice treated with MK-591. We observed that mice receiving MK-591 had a statistically significant reduction in the steady state levels of three of the four components of the γ-secretase complex, PS1, Pen-2 and APH-1 (FIG. 2A, B). Although we observed a reduction for Nicastrin, it did not reach statistical significance (p=0.06). Furthermore, quantitative real time RT-PCR analyses revealed that in the same mice the mRNA levels of these four proteins were significantly reduced (FIG. 2C). By contrast, no change was observed in the BACE-1 mRNA levels (FIG. 2C). These results indicate that FLAP inhibition with MK-591 influences the metabolism of APP in part through a reduction in the steady state levels of three of the four components of the γ-secretase complex, PS1, Pen-2 and APH-1.

Flap Blockade does not Affect Aβ Catabolic Pathways

Since the final amount of Aβ assayed is the result of production and degradation, we next analyzed two of the major proteases involved in its catabolism: i.e., insulin-degrading enzyme (IDE), and neprilysin (Leissring M A et al. Neuron 2003, 40:1087-1093). Steady state levels of both proteins measured by western blot analysis were similar between the two groups of mice: control mice and in mice treated with MK-591 (not shown). A similar result was observed when we measured levels of apolipoprotein E (apoE), which has been implicated in the clearance of Aβ from the CNS by acting as a chaperone (Guenette SY Neuromolecular Med 2003, 4:147-160). This indicates that treatment with MK-591 does not affect Aβ catabolic pathways.

### FLAP Blockade Modulates Neuroinflammation

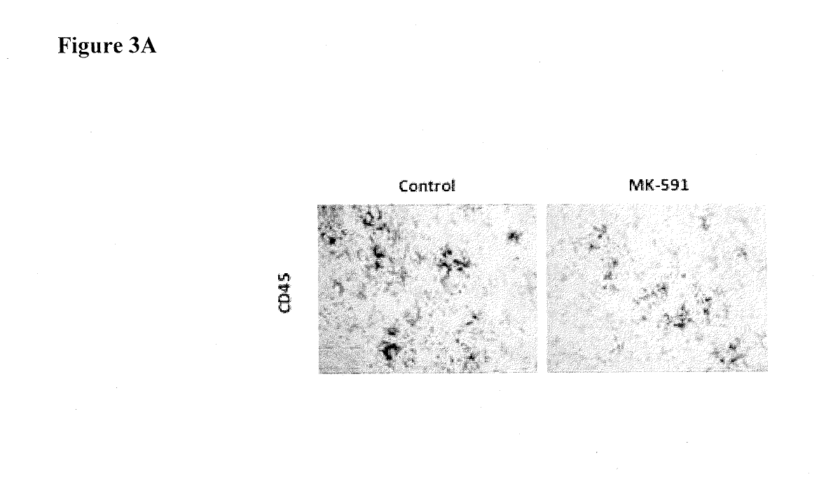

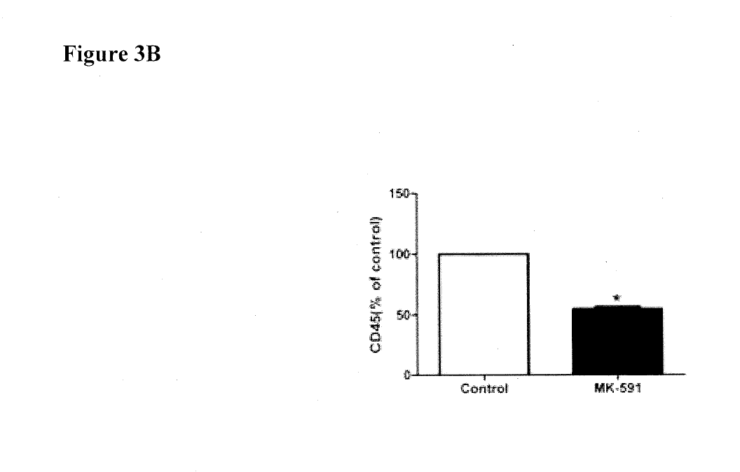

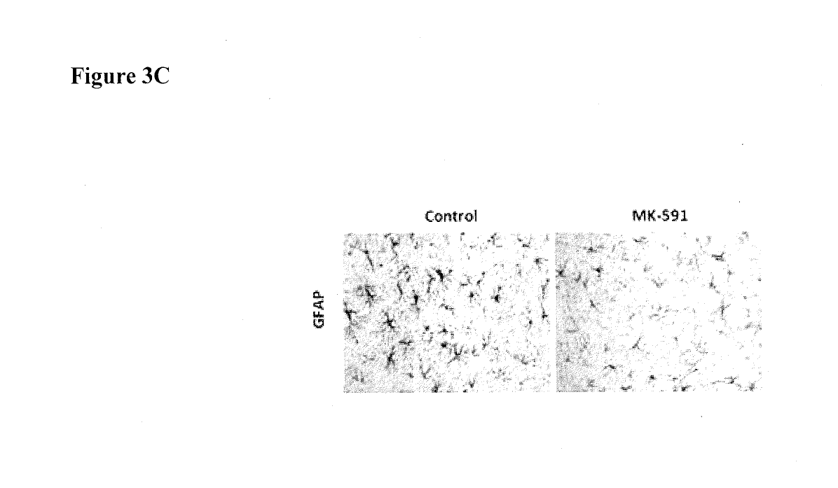

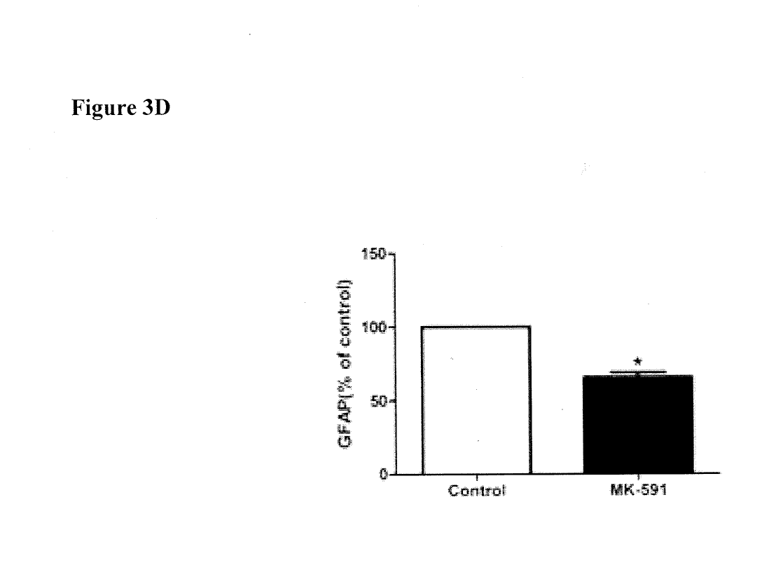

Since neuroinflammation is also an important feature of this AD-like amyloidosis model (Yao Y et al. J Neuroinflammation 2004, 1:21), we next investigated the effect of FLAP pharmacologic blockade on microglia and astrocytes activation. As shown in FIG. 3, mice receiving MK-591 had a significant decrease in the immunoreactivity for CD45, a marker of microgliosis, and GFAP, a marker for astrogliosis, indicating that treatment with MK-591 modulates neuroinflammation.

### MK-591 Affects CREB but Not Sp1

The above-described data suggests that MK-591 by blocking FLAP regulates the γ-secretase complex expression at the transcription level. Previous studies have shown that 5LO activation by producing HETEs can influence cAMP-response element binding protein (CREB), a transcriptional factor that regulates gene expression (Chu J et al. Ann Neurol 2011, 69:34-46). Compared with mice on placebo, we found that mice treated with MK-591 showed a statistically significant decrease in the steady state levels of total CREB and its phosphorylated form at Ser-133. However, MK-591 did not significantly affect the steady state levels of Sp1, another transcription factor (FIG. 2D, E).

### In Vitro Studies

### MK-591 Influences Aβ Formation in a γ-Secretase-Depenent Manner

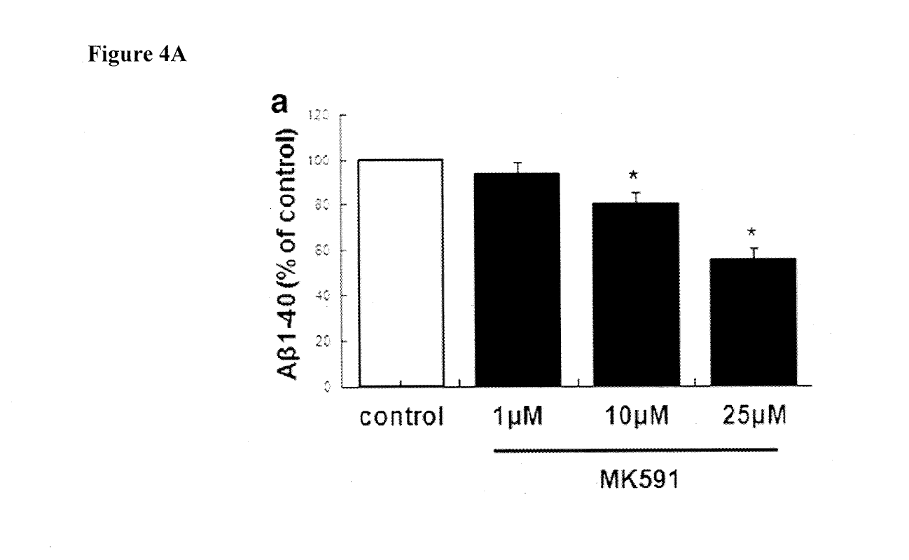

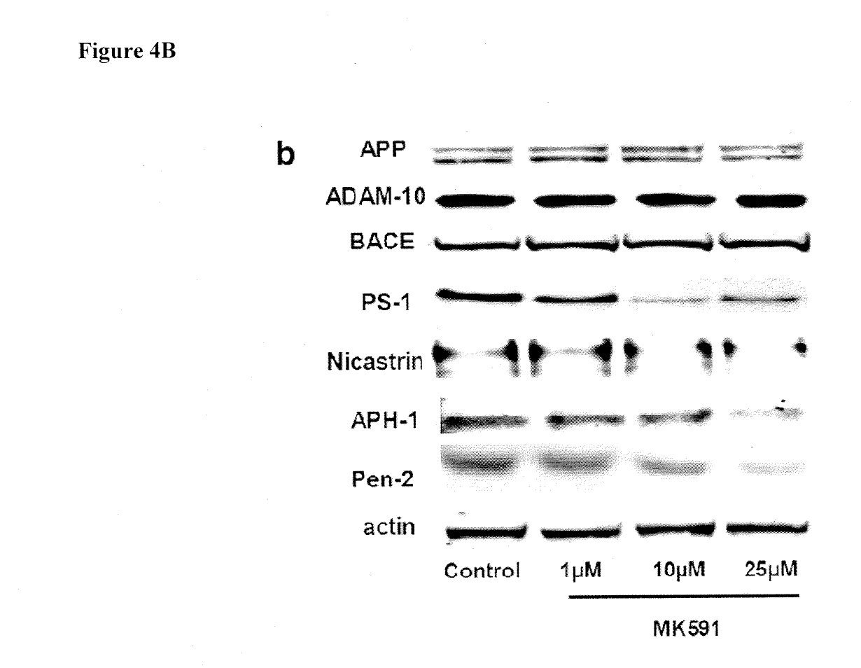

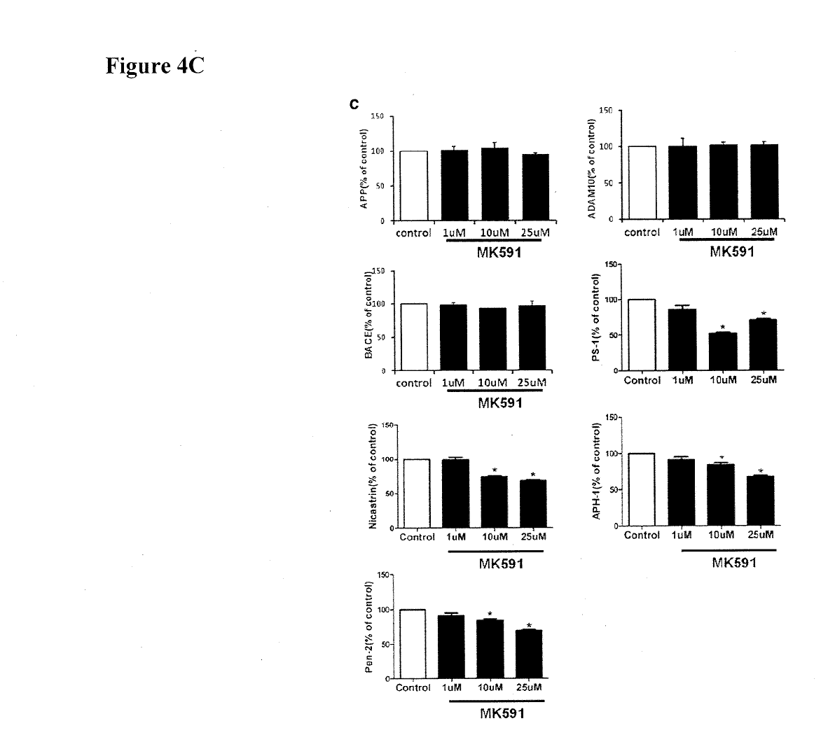

To further confirm our ex vivo observations detailed above, the following in vitro experiments were conducted. N2A-APPswe cells were incubated with MK-591 for 24 hours at different concentrations (1, 10, 25 μM) or vehicle. At the end of this period, conditioned media showed that compared with control, MK-591 dose-dependently reduced Aβ1-40 formed by these cells (FIG. 4A). This reduction was associated with a significant decrease in the steady state levels of the PS1, nicastrin, APH-1 and Pen-2 proteins, the four components of the γ-secretase complex (FIG. 4B, C). By contrast, MK-591 did not influence the protein levels for APP, BACE-1 and ADAM-10 (FIG. 4B, C). These results indicate that MK-591 influences Aβ formation in a γ-secretase-dependent manner.

### MK-591 Influences CREB but not Sp1

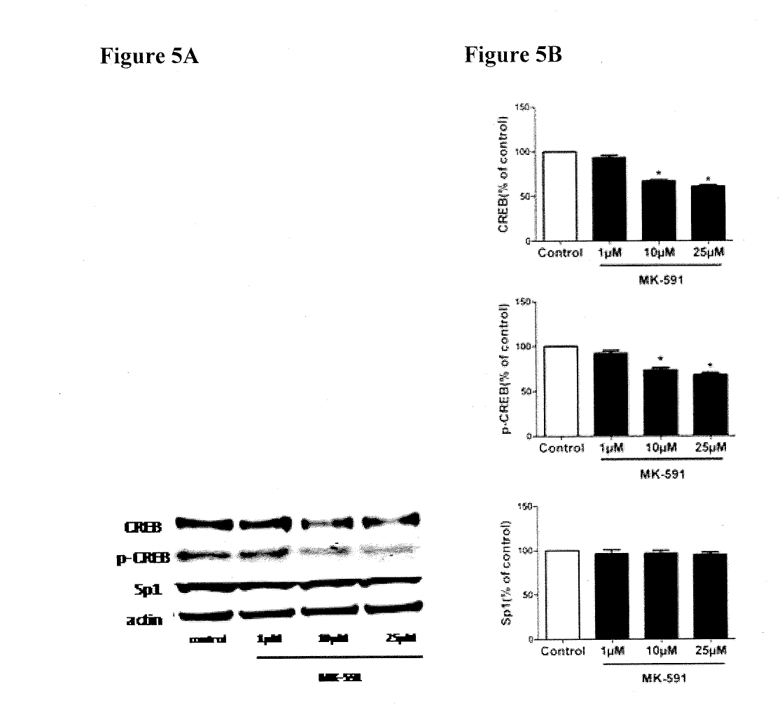

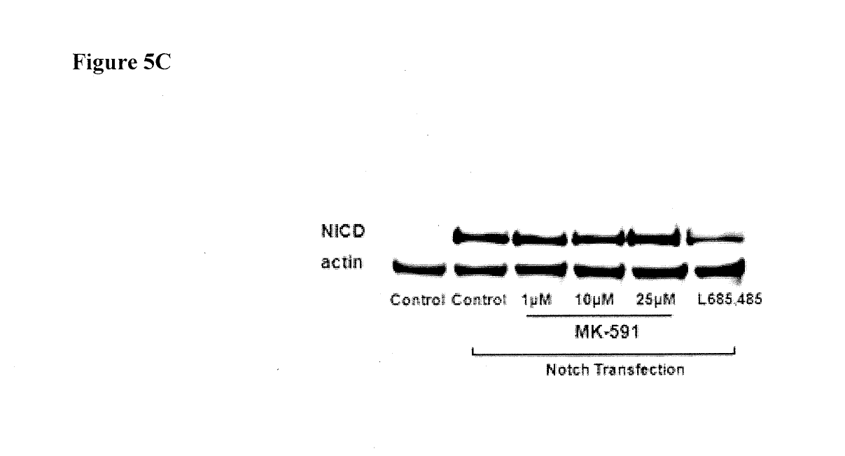

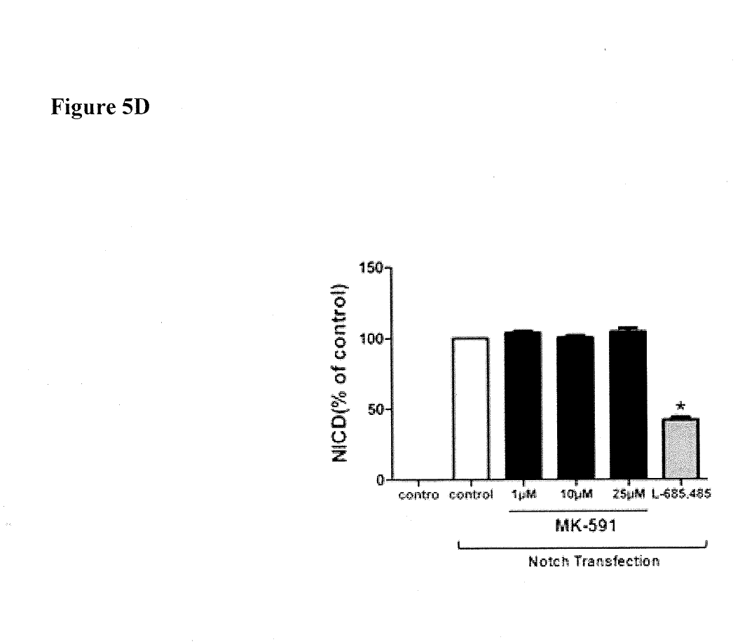

Similar to the in vivo experiments, we also observed that incubation of MK-591 with N2A-APP cells resulted in a significant decrease in the expression levels of CREB and p-CREB. By contrast, the presence of the drug did not induce any significant alteration in the levels of another transcription factor, i.e., Sp1 (FIG. 5A, B).

MK-591 does not Affect Notch Signalling

Since Notch is another possible substrate for γ-secretase proteolytic activity, we next tested whether this pathway was affected by the MK-591 treatment. To this end, we assess the effect of MK-591 on γ-secretase-mediated cleavage of Notch. N2A-APPswe cells were transfected with Myc-tagged mAE-Notch-1 complementary DNA and after incubating them with MK-591 at the same concentrations which reduced γ-secretase components, we assessed the expression levels of Notch intracellular domain (NICD) by western blot analysis. As shown in FIGS. 5C and D no significant difference in the levels of NICD was observed between cells with and without MK-591 treatment. By contrast, when the specific γ-secretase inhibitor L685,458 was used, a significant reduction in NICD levels was detected (FIG. 5C, D).

### Example 2

### Genetic Absence and Pharmacological Blockade of FLAP Ameliorates Cognition

### A. Procedures

### Animals

The 3xTg mice harboring a mutant amyloid precursor protein (APP; KM670/671NL), a human mutant PS1 (M146V) knockin, and tau (P301L) transgenes, wild type mice (WT), and mice genetically deficient for FLAPKO (FLAPKO) used in this study were reported previously (Oddo S et al. Neuron 2003, 39:409-421; Byrum R S et al. J Exp Med 1997, 185:1065-1075). All the animals were backcrossed 10 times on the same genetic background C57BL6/SJL. The FLAPKO mice were crossbred several times with 3xTg mice to obtain founder animals (3xTg/FLAPKO), which were then crossed with each other. The animals from these crosses were used for the studies. The animals were kept in a pathogen-free environment, on a 12 hour light/dark cycle, and fed a normal chow and water ad libitum. Animals underwent behavioral testing at two different age groups (6-8 months and 12-14 months). Starting at 4-5 months of age, a separate group of five month old 3xTg mice were also randomized to receive MK-591 (40 mg/kg weight) in their chow diet, while some were sacrificed at 6 months of age for electrophysiology studies, others were kept on the same diet until they were 13-14 months old. Considering that each mouse eats approximately 5 g/day of the chow diet formulated for 320 mg MK-591 per kg diet (Harlan Teklad, Wis., USA), the final dose of the drug intake is about 40 mg/kg weight/day. At sacrifice, the mice were perfused with ice-cold 0.9% PBS containing EDTA (2 mmol/L) pH 7.4. Brain was removed, gently rinsed in cold 0.9% PBS and immediately dissected in two halves. One half was immediately stored at −80° C. for biochemistry; the other half was fixed in 4% paraformaldehyde in PBS, pH 7.4 for immunohistochemistry studies.

### Behavioral Tests

All the animals were handled for at least 3-4 days prior to testing. They were tested in random order and the experimenter conducting the tests was unaware of the genotype of treatment.

### Y-maze

The Y-maze apparatus consisted of three arms 32 cm (long)×10 cm (wide) with 26-cm walls (San Diego Instruments). Testing was always performed in the same room and at the same time to ensure environmental consistency as previously described (Chu J et al. J Neuroinflammation 2012, 9:2094-2099).

### Fear-Conditioning

Two weeks before sacrifice, fear conditioning experiments were performed following methods described previously (Chu J et al. J Neuroinflammation 2012, 9:2094-2099; Yang H et al. Biol Psychiatry 2010, 68:922-929; Zhuo et al. Curr Alzheimer Res 2010, 7:140-149). Tests were conducted in a conditioning chamber equipped with black methacrylate walls, a transparent front door, a speaker and grid floor (Start Fear System; Harvard Apparatus).

### B. Results

Mice genetically deficient in FLAP and mice pharmacologically inhibited for FLAP with MK-591 both showed a significant improvement in their memory performance as early as 6 months of age, as demonstrated in the Y-maze paradigm which assesses working memory in the mice through the recording of spontaneous alternation behavior. These same groups of mice also showed improvements in their learning memory ability, as evaluated by the fear conditioning test paradigm.

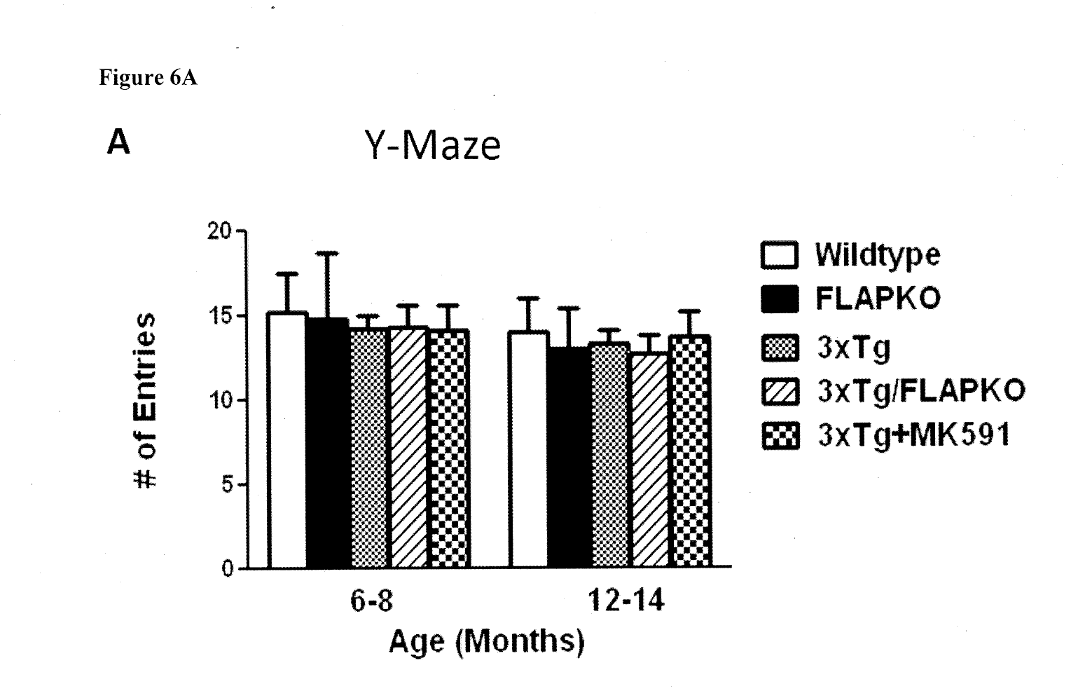

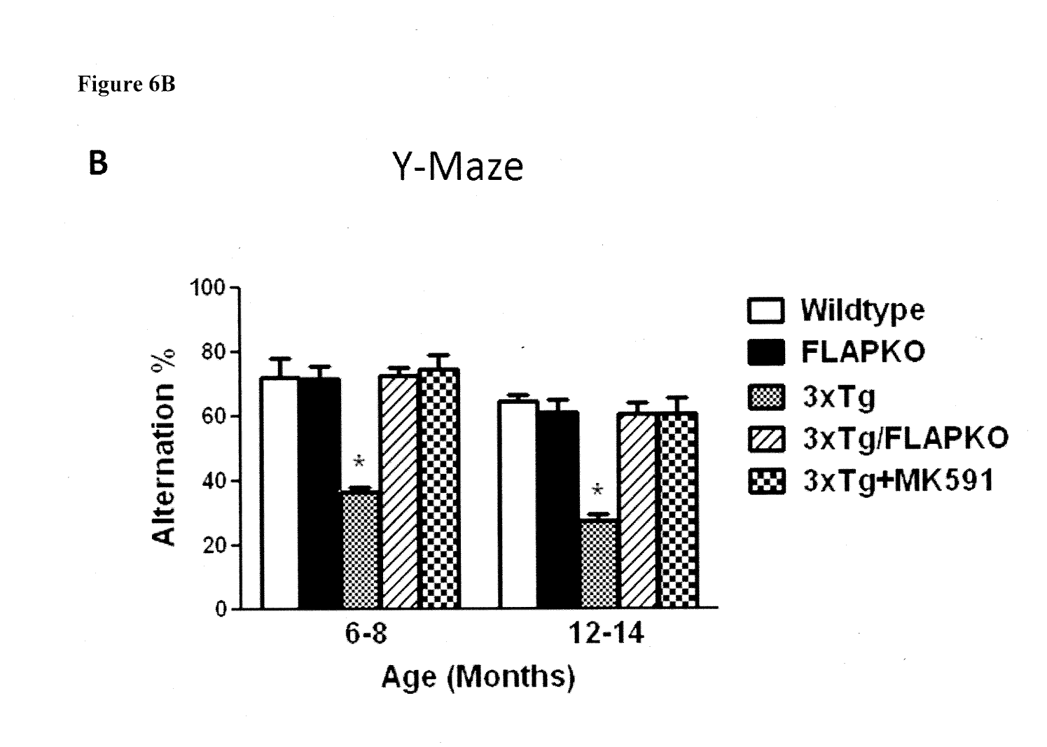

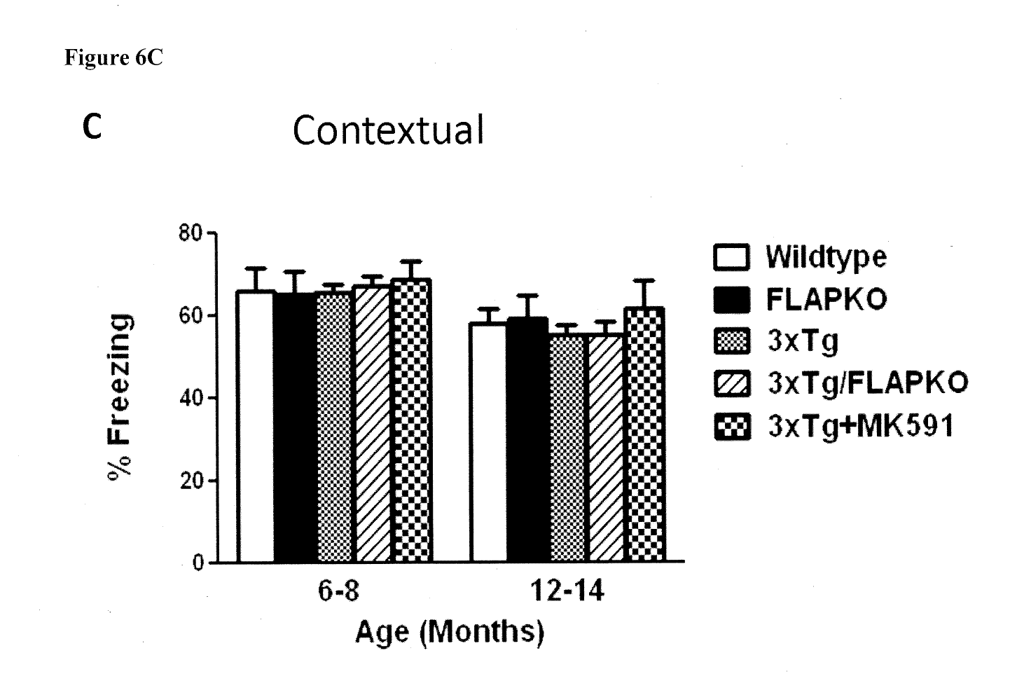

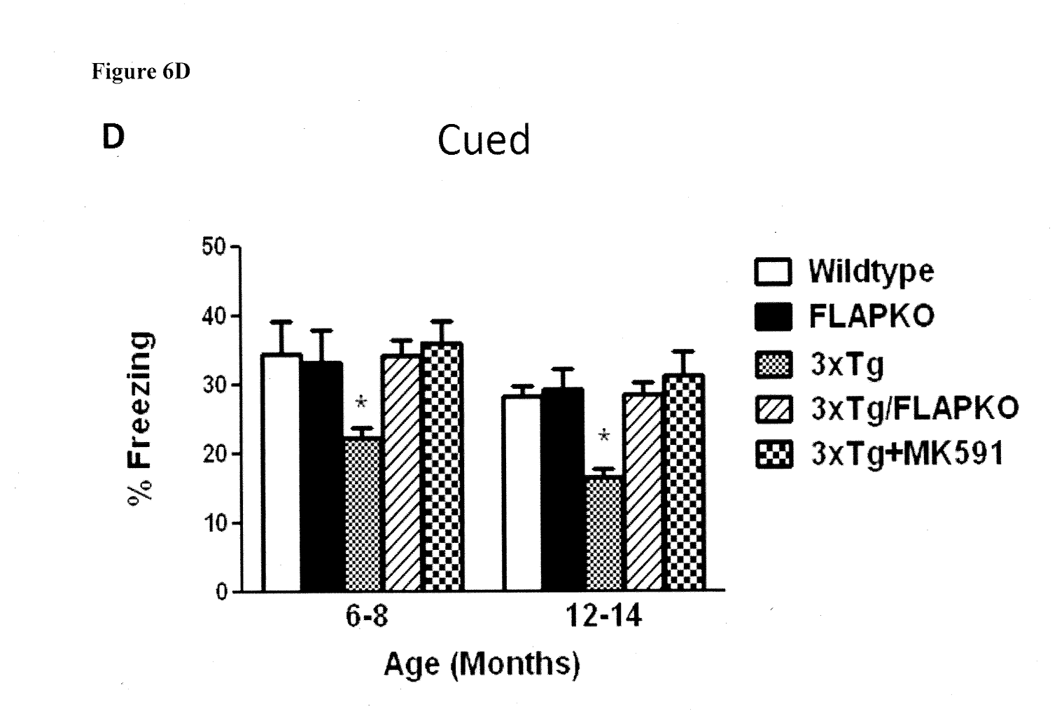

To evaluate the effect of FLAP genetic absence and pharmacological inhibition on behavior, mice were initially tested in the Y-maze at two different ages: 6-8 and 12-14 months old. Initially, no differences were noted among the control and treated groups when considered in regard to their general activity as assessed by the total number of arm entries at both ages (FIG. 6A). However, when the number of alternations was counted in the same test, the 3xTg mice had a much lower number of alternations resulting in a significantly lower percentage in comparison with the wild type and FLAPKO mice. By contrast, compared with the 3xTg mice, the 3xTg-FLAPKO and 3xTg receiving MK-591 had a greater number of alternations resulting in a significantly higher percentage in both age groups, indicating an improvement in their working memory (FIG. 6B).

Next, mice underwent fear conditioning testing. No differences were observed during the training session among the different groups (not shown), and a similar result was observed when they were assessed in the contextual fear conditioning recall paradigm (FIG. 6C). By contrast, in the cued phase of the fear conditioning testing while we observed that FLAPKO and wild type mice exhibited similar levels of freezing at both ages, 3xTg mice had significantly lower freezing percentages which were normalized in the 3xTg-FLAPKO and 3xTg mice treated with MK-591 (FIG. 6D).

### Example 3

### Unavailability of FLAP Reduces Brain Aβ Level and Deposition, and Tau Phosphorylation

### A. Procedures

### Immunoblot Analyses

Primary antibodies used in this example are summarized in Table 1. Proteins were extracted in EIA buffer containing 250 mM Tris base, 750 mM NaCl, 5% NP-40, 25 mM EDTA, 2.5% Sodium Deoxycholate, 0.5% SDS and an EDTA-free protease and phosphatase inhibitors cocktail tablet (Roche Applied Science), sonicated, centrifuged at 13,000 rpm for 45 min at 4° C., and supernatants used for immunoblot analysis, as previously described (Chu J et al. J Neuroinflammation 2012, 9:2094-2099, Yang H et al. Biol Psychiatry 2010, 68:922-929). Total protein concentration was determined by using BCA Protein Assay Kit (Pierce, Rockford Ill.). Samples were electrophoretically separated using 10% Bis-Tris gels or 308% Tris-acetate gel (Bio-Rad, Richmond Calif.), according to the molecular weight of the target molecule, and then transferred onto nitrocellulose membranes (Bio-Rad). The samples were blocked with Odyssey blocking buffer for 1 hr; and then incubated with primary antibodies overnight at 4° C. After three washing cycles with T-TBS, membranes were incubated with IRDye 800CW or IRDye 680CW-labeled secondary antibodies (LI-COR Bioscience, NE) at 22° C. for 1 hr. Signals were developed with Odyssey Infrared Imaging Systems (LI-COR Bioscience). Actin was always used as an internal loading control.

### Sarkosyl Insolubility Assay

The assay for insoluble tau was performed as described previously (Andorfer C et al. J Neurochem 2003, 86:582-590). Briefly, ultracentrifugation and sarkosyl extraction (30 min in 1% sarkosyl) was used to obtain soluble and insoluble fractions of tau. Insoluble fractions were washed one time with 1% sarkosyl, then immunoblotted with HT-7 antibody (Table 1).

TABLE 1
Antibodies used in this study

| Antibody | Immunogen | Host | Application | Source |
|---|---|---|---|---|
| 4G8 | aa 18-22 of human beta amyloid (VFFAE) | Mouse | IHC | Covance |
| APP | aa 66-81 of APP (N-terminus) | Mouse | WB | Millipore |
| PS-1 | aa around valine 293 of human presenilin | Rabbit | WB | Cell Signaling |
| Nicastrin | aa carboxy-terminus of human Nicastrin | Rabbit | WB | Cell Signaling |
| APH-1 | Synthetic peptide from hAPH-1a | Rabbit | WB | Millipore |
| Pen-2 | aa N-terminal of human and mouse Pen-2 | Rabbit | WB | Invitrogen |
| HT-7 | aa 159-163 of human tau | Mouse | WB | Pierce |
| AT-270 | Peptide containing phosphor-T181 | Mouse | WB | Pierce |
| PHF-13 | Peptide containing phosphor-Ser396 | Mouse | WB | Cell Signaling |
| PHF-1 | Peptide containing phosphor-Ser396/S404 | Mouse | WB | Dr. P. Davies |
| GFAP | aa spinal chord homogenate of bovine origin | Mouse | WB | Santa Cruz |
| CD45 | Mouse thymus or spleen | Rat | WB | BD Pharmingen |
| SYP (H-8) | aa 221-313 of SYP of human origin | Mouse | WB, IHC | Santa Cruz |
| PSD95 | Purified recombinant rat PSD-95 | Mouse | WB, IHC | Thermo Scient |
| MAP2 | Bovine brain microtubule protein | Rabbit | WB, IHC | Millipore |
| GSK3α/β | aa 1-420 full length GSK-3β of Xenopus origin | Mouse | WB | Millipore |
| p-GSK3α/β | aa around Ser21 of human GSK-a | Rabbit | WB | Cell Signaling |
| JNK | aa of human JNK | Rabbit | WB | Cell Signaling |
| SAPK/JNK | aa of recombinant human JNK2 fusion protein | Rabbit | WB | Cell Signaling |
| P- | aa Thr183/Tyr185 of human SAPK/JNK | Mouse | WB | Cell Signaling |
| SAPK/JNK | | | | |
| Cdk5 | aa C-terminus of Cdk5 of human origin | Rabbit | WB | Santa Cruz |
| P 35/25 | aa C-terminus of p35/25 of human origin | Rabbit | WB | Santa Cruz |
| PP2A | aa 295-309 of catalytic subunit of human protein | Mouse | WB | Millipore |
| | phosphatase 2A. Clone 1D6. | | | |
| Actin | aa C-terminus of Actin of human origin | Goat | WB | Santa Cruz |

IHC: Immunohistochemistry
WB: Western blot

### Biochemical Analyses

Mouse brain homogenates were sequentially extracted initially in RIPA for the Aβ1-40 and 1-42 soluble fractions, then in formic acid for the Ab 1-40 and 1-42 insoluble fractions, and then assayed by a sensitive sandwich ELISA kits (WAKO Chem.), as previously described (Chu J et al. Am J Pathol 2011, 178:1762-1769; Firuzi O et al. FASEB J2008, 22:1169-1178).

### Immunohistochemistry

Primary antibodies used in this study are listed in Table 1. Immunostaining was performed as reported previously (Chu J et al. Am J Pathol 2011, 178:1762-1769; Firuzi O et al. FASEB J2008, 22:1169-1178; Yang H et al. Biol Psychiatry 2010, 68:922-929). First, serial 6 μm-thick coronal sections were mounted on 3-aminopropyl triethoxysilane-coated slides. Every eigth section from the habenular to the posterior commissure (8-10 sections per animal) was examined using unbiased stereological principles. The sections for testing Aβ were deparaffinized, hydrated and pretreated with formic acid (88%) and subsequently with 3% H₂O₂ in methanol. The sections for Synaptophysin (SYP), Postsynaptic Density Protein 95 (PSD95) and Microtubule associated protein-2 (MAP-2) were deparaffinized, hydrated, subsequently treated with 3% H₂O₂ in methanol, and then treated with citrate (10 mM) or IHC-Tek Epitope Retrieval Solution (1HC World, Woodstock, Md.) for antigen retrieval. Sections were blocked in 2% fetal bovine serum and then incubated with primary antibody overnight at 4° C. The following day, sections were incubated with biotinylated anti-mouse immunoglobulin G (Vector Laboratories, Bulingame, Calif.) and then developed by using the avidin-biotin complex method (Vector Laboratories) with 3,3′-diaminobenzidine as a chromogen. Light microscopic images were used to calculate the area occupied by Aβ immunoreactivity, and the cell densities of GFAP- and CD45-immunopositive reactions by using the software Image-Pro Plus for Windows version 5.0 (Media Cybernetics, Bethesda, Md.). The threshold optical density that discriminated staining from background was determined and held constant for all quantifications. The area occupied by Aβ immunoreactivity was measured by the software and divided by the total area of interest to obtain the percentage area of immunoreactivity.

### B. Results

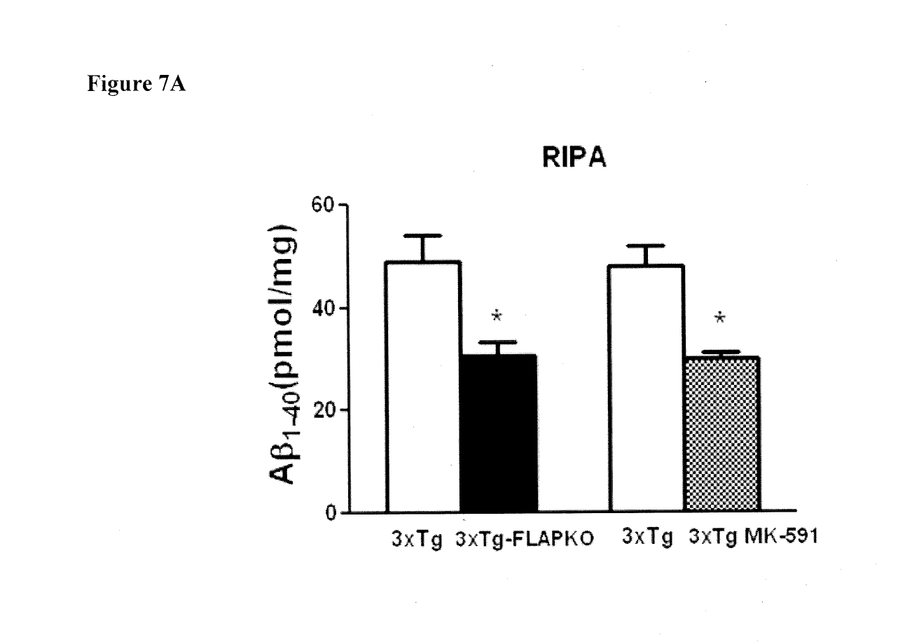

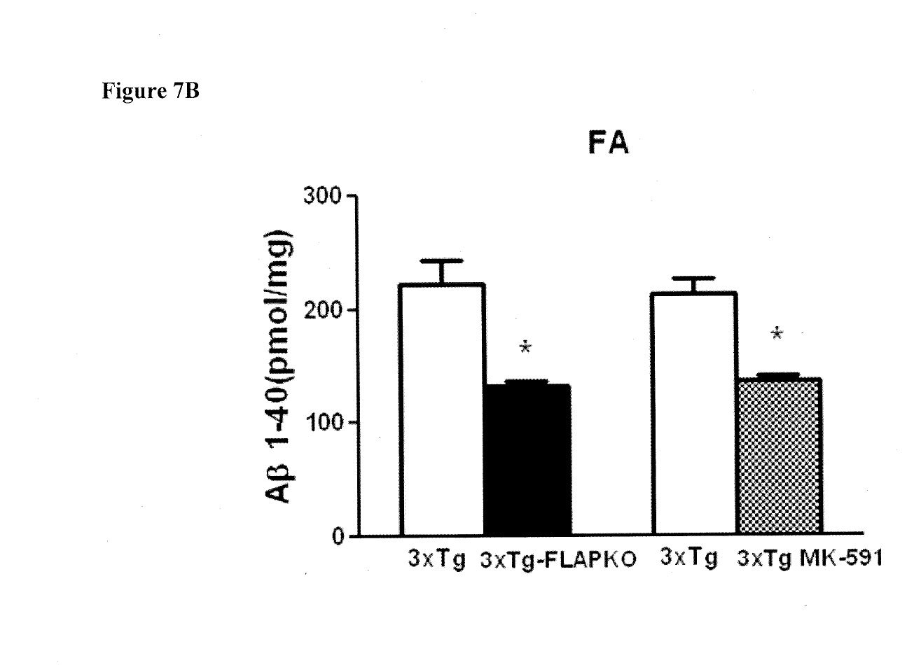

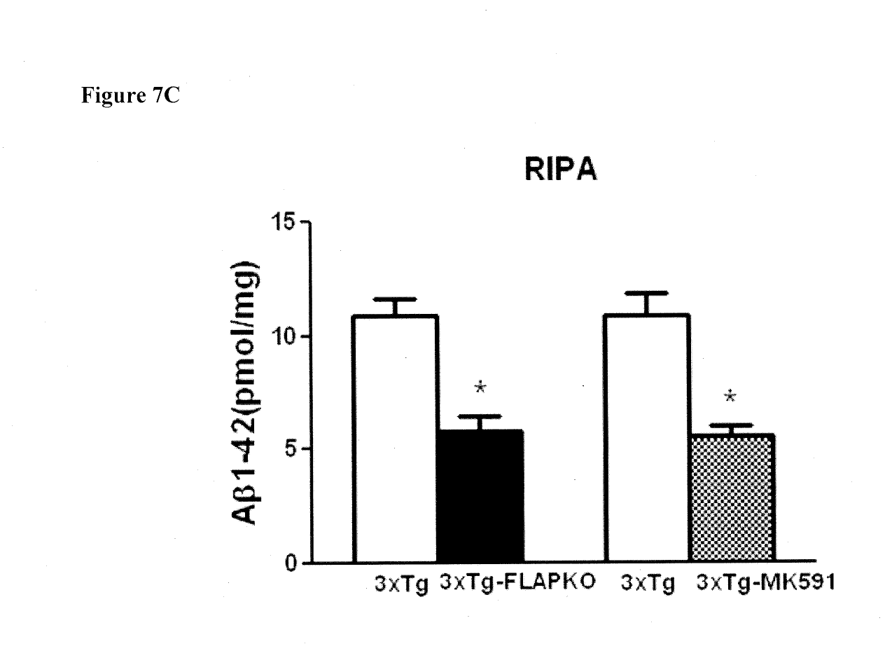

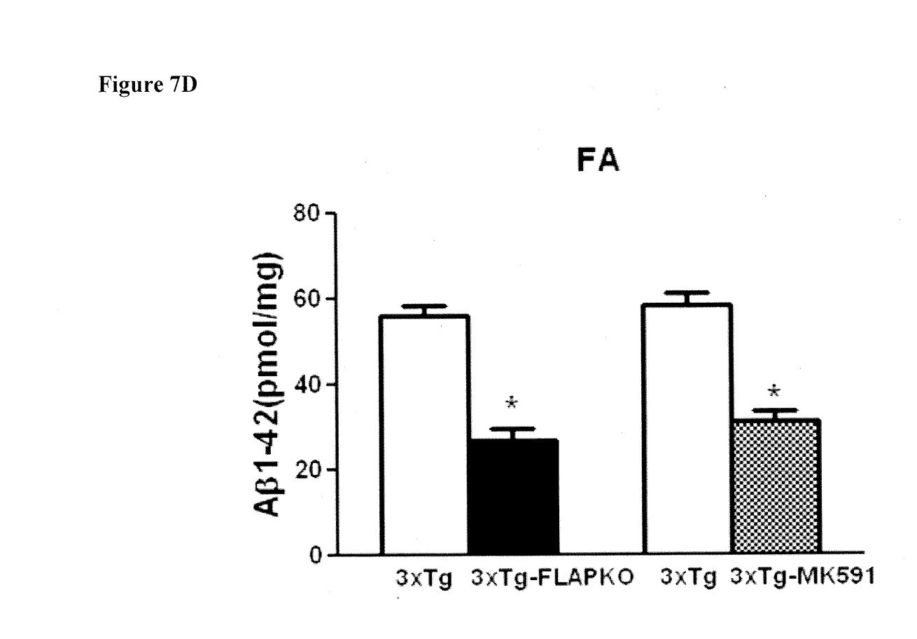

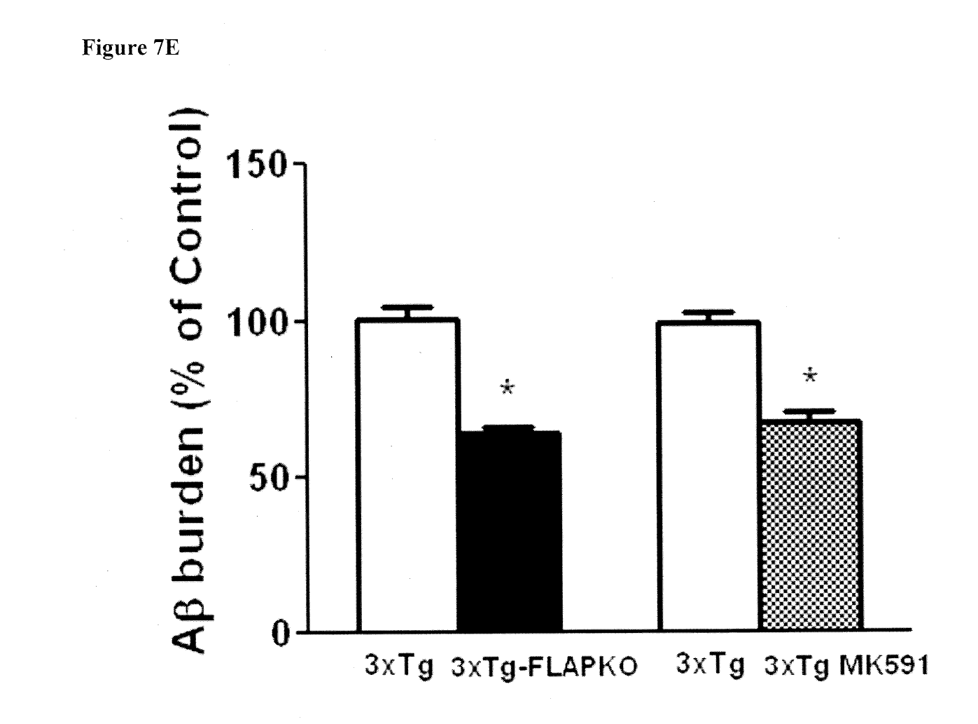

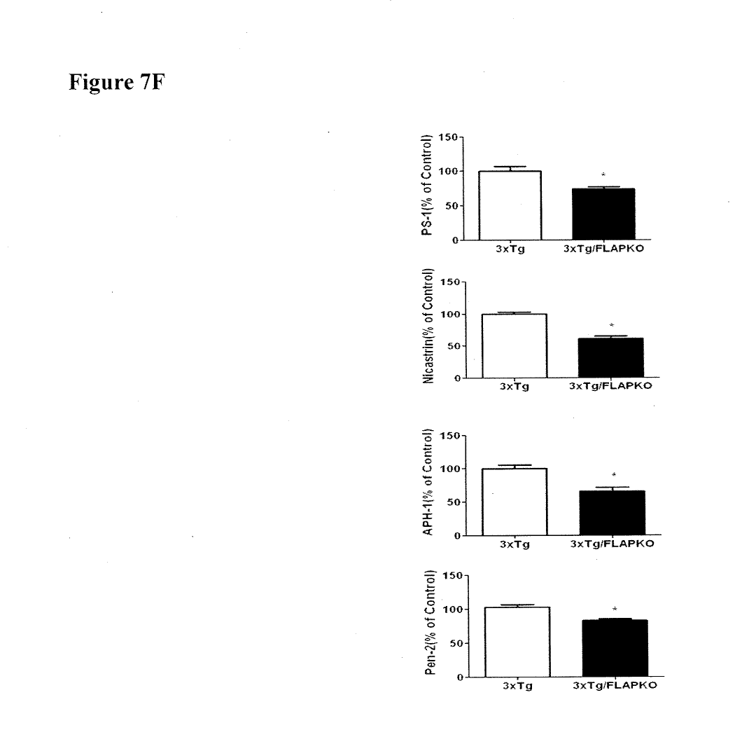

A week after completion of the behavior tests (14 months of age), mice were sacrificed, brains were harvested and assayed for levels and deposition of Aβ. As shown in FIG. 7A-D, we observed that 3xTg-FLAPKO and 3xTg mice receiving MK-591 displayed a significant decrease in the amount of RIPA-soluble and formic-acid soluble Aβ1-40 and 1-42. Confirming the ELISA data, we found that the same conditions led to a significant decrease in Aβ deposition in their brains (FIG. 7E). To investigate the mechanism responsible for this change, we assessed the steady-state levels of APP along with its cleavage products. As shown in FIG. 7F-G, no differences were noted for total APP. However, compared with controls, the 3xTg-FLAPKO and 3xTg treated with MK-591 had a significant decrease in the steady-state levels of the 4 components of the γ-secretase complex, PS1, nicastrin, Pen-2 and APH-1 (FIG. 7F-G). No differences were observed in the α-secretase (ADAM-10) and β-secreatse (BACE-1) pathways between the two groups (not shown). Also no changes were detected in the steady-state levels of neprilysin and insulin-degrading enzyme, which are involved in Aβ degradation, nor in apolipoprotein E, and Aβ chaperon, that could justify the decrease in Aβ (not shown).

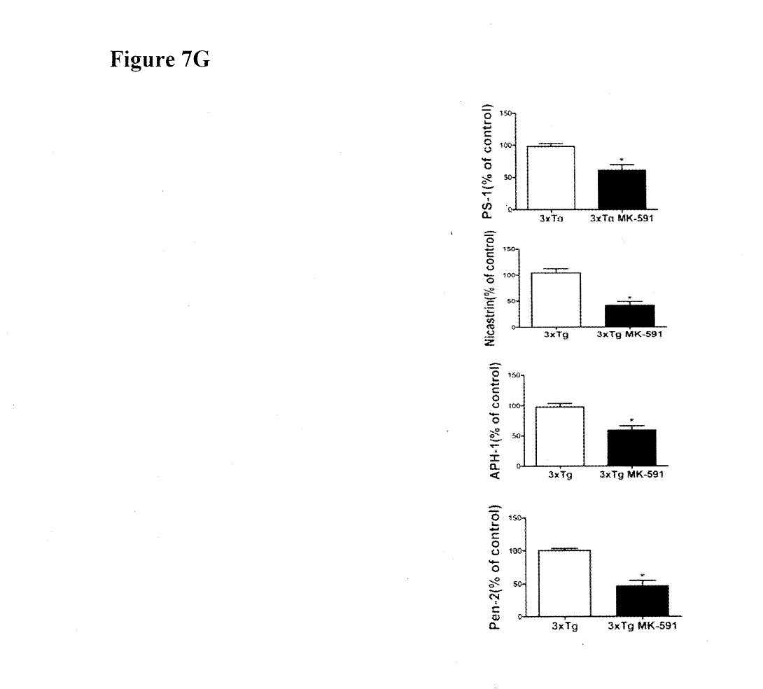

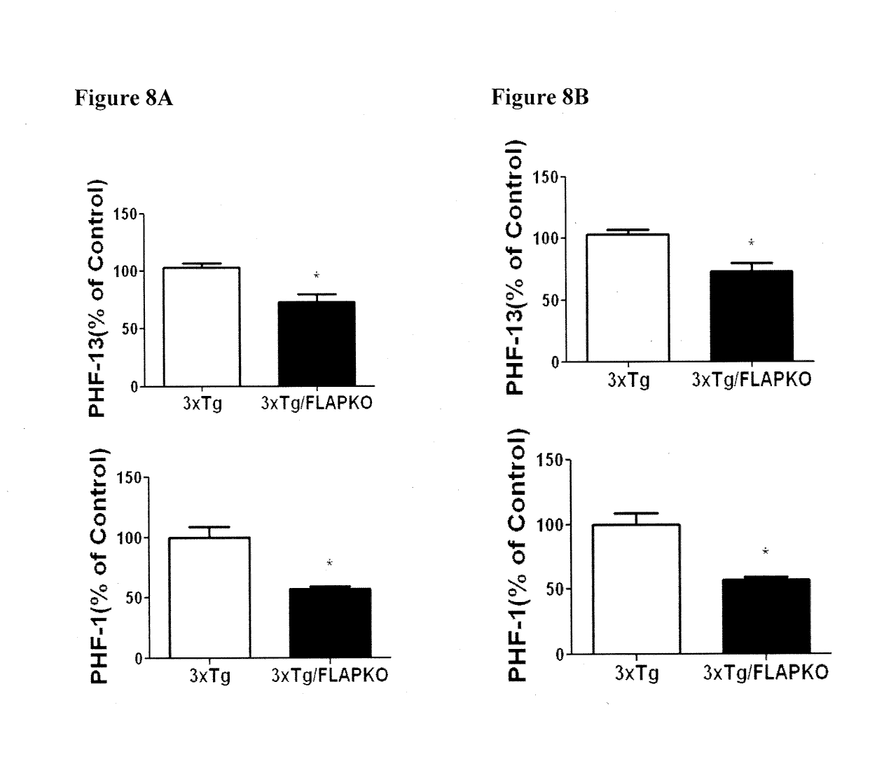

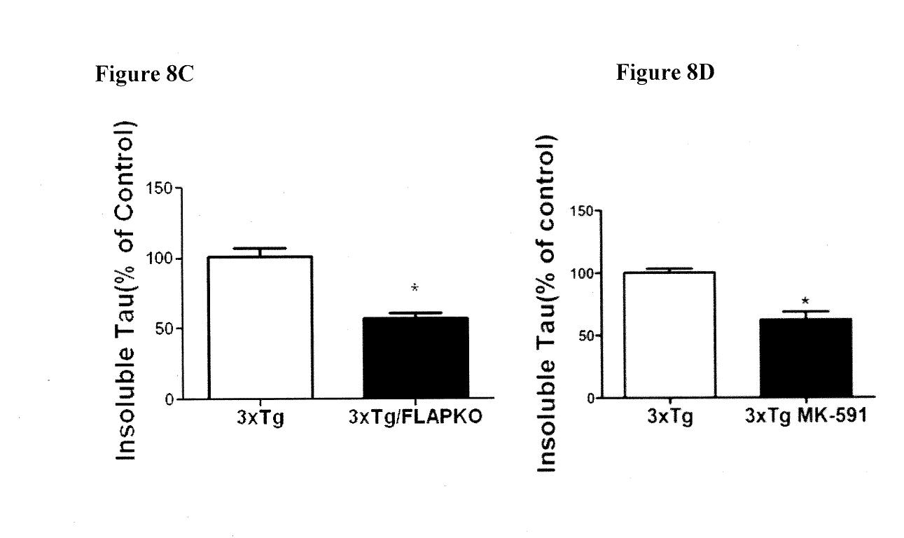

The effect of FLAP knockout and pharmacological blockade on tau metabolism was also explored. As shown in FIG. 8, while no changes in the levels of total soluble tau were observed among the groups, compared with the 3xTg, the 3xTg-FLAPKO and 3xTg mice treated with MK-591 had a significant decrease in its phosphorylated forms at epitopes 5396, as recognized by the antibody PHF-13, and epitopes S396/S404 as recognized by the antibody PHF-1 (FIG. 8A-B). By contrast, no changes were detected for the phosphorylation site recognized by the antibody AT270 (T181). Additionally, compared with 3xTg, we observed that 3xTg-FLAPKO and 3xTg receiving MK-591 had a significant reduction in the levels of insoluble tau (FIG. 8C-D).

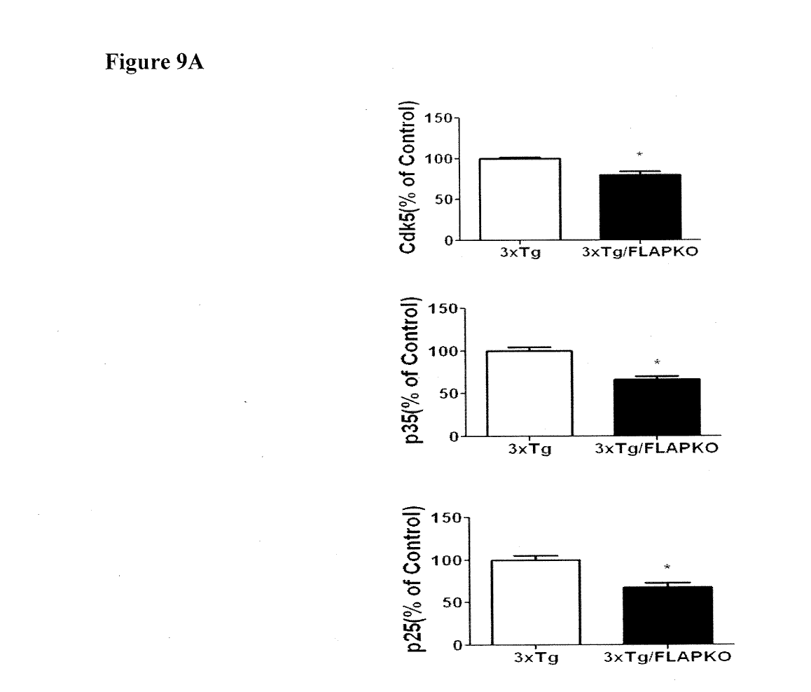

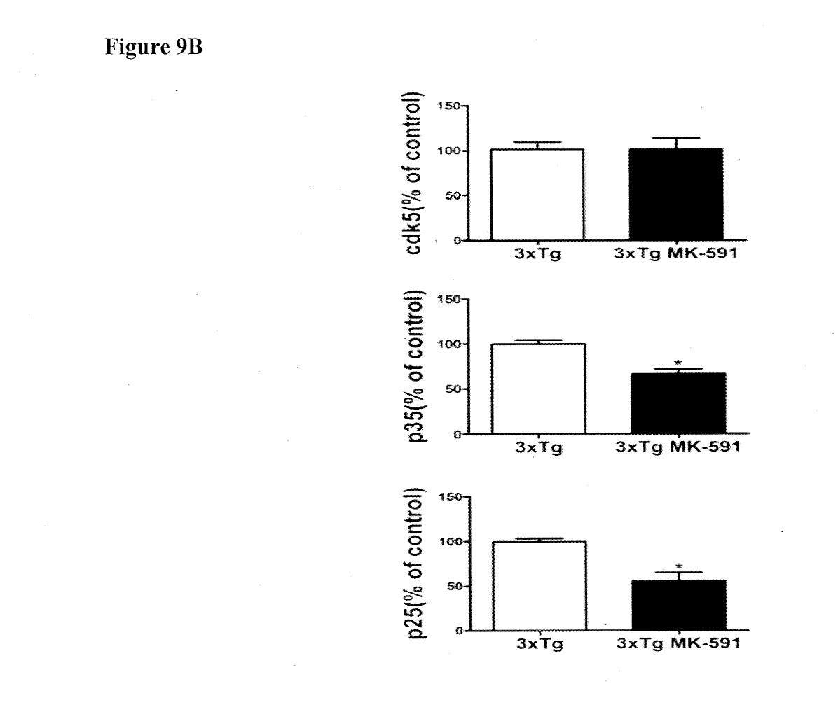

To investigate the molecular mechanism responsible for the hypophosphorylation of tau, we next assayed some of the kinases which are considered major regulators of post-translational modification of this protein. First, we did not observe any significant differences among the different groups in the steady-state levels of total and phosphorylated forms of GSK3-α and GSK3-β, SAPK/JNK and total and phosphorylated SAPK/JNK (FIG. 9A-B). However, we found that compared with controls, 3xTg-FLAPKO and 3xTg receiving MK-591 had a statistically significant decrease in levels of Cdk5 kinase and its coactivators p35 and p25 (FIG. 9A-B). No changes in the levels of phosphatase—A2 (PPA2) were detected among the different groups of mice (FIG. 9A-B).

### Example 4

### Genetic Absence and Pharmacological Blockade of FLAP Ameliorates Synaptic Integrity

### A. Procedures

### Electrophysiology

Six month old mice (n=# slices/# animals): WT (n=23/8); 3xTg (n=21/7); 3xTg-FLAPKO (n=21/6); 3xTg plus MK-591 (n=10/3) were sacrificed by rapid decapitation and brains were put into ice-cold artificial cerebral spinal fluid (CSF) in which sucrose (248 mM) was substituted for NaCl. Transverse hippocampal slices (400 μm thick) were cut using a Vibratome 3000 plus (Vibratome, Bannockburn, Ill.) and placed in ACSF (124 mM NaCl, 1.5 mM KCl, 2 mM NaH₂PO₄, 2.5 mM CaCl₂, 2 mM MgSO₄, 10 mM dextrose, and 26 mM NaHCO₃) at room temperature to recover for 1 hr bubbled with 95% O₂/5% CO₂. Slices were transferred to a recording chamber (Warner Instruments, Hamden Conn.) and continuously perfused with ACSF at 1.5-2.0 ml/min flow, bubbled with 95% O₂/5% CO₂, and maintained by an in-line solution heater (TC-324; Warner Instruments) at 32-34° C. We recorded field excitatory postsynaptic potentials (fEPSPs) from the CAl stratum radiatum by using an extracellular glass pipette (3-5 MS2) filled with ACSF. Schaffer collateral/commissural fibers in the stratum radiatum were stimulated with a bipolar tungsten electrode placed 200-300 μm from the recording pipette. Stimulation intensities were chosen to produce a fEPSP that was ⅓ of the maximum amplitude, based on an input/output curve using stimulations of 0-300 μA, in increments of 20 μAs. Paired-pulse facilitation experiments were performed using a pair of stimuli of the same intensity delivered 20, 50, 100, 200 and 100 ms apart. Baseline was recorded for 20 mins prior to tetanization with pulses every 30 seconds. LTP at CA3-CA1 synapses was induced by four trains of 100 Hz stimulation delivered in 20 second intervals. Recordings were made every 30 seconds for 3 hours following tetanization. The fEPSP rise/slope (mV/ms) between 30 and 90% was measured offline using Clampfit 10.3 (Molecular Devices, LLC) and normalized to the mean rise/slope of the baseline. Slices were eliminated if an unstable baseline was produced or if the normalized rise/slope dropped more than 20-50 mV/ms in an approximately 10 min period. All the tests were performed by an experimenter who was unaware of the different genotypes and treatment.

### Data Analysis

One-way analysis of variance (ANOVA), unpaired Student's t-test (two-sided) and Bonferroni multiple comparison tests were performed using Prism 5.0 (GraphPad Software, La Jolla, Calif.). All data are presented as mean +/− standard error of the mean. Significance was set at p<0.05.

### B. Results

We observed that mice genetically deficient in FLAP (3xTg-FLAPKO) and 3xTg mice treated with MK-591 had a significant increase in three distinct protein markers of synaptic integrity (i.e. Synaptophysin, PSD95 and MAP2).

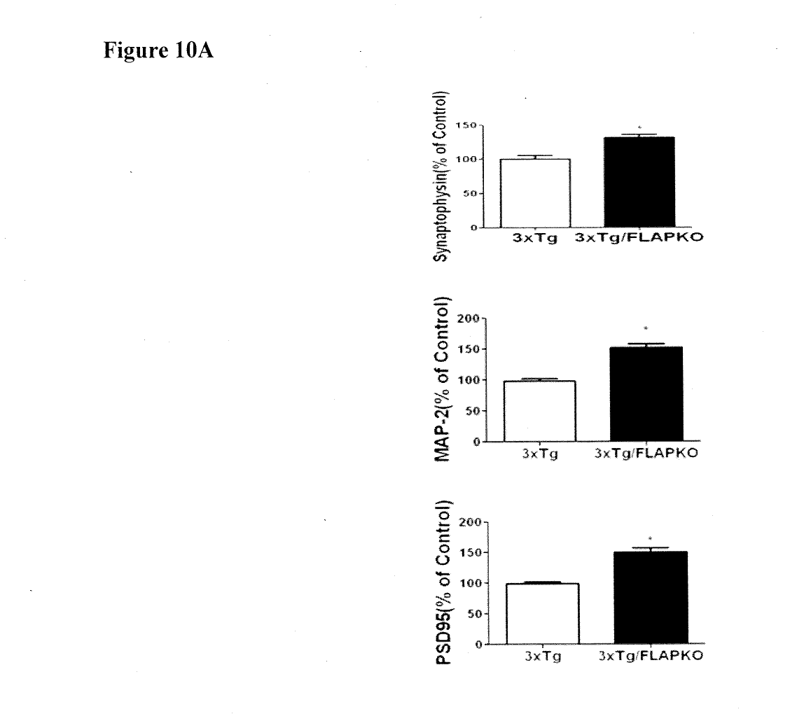

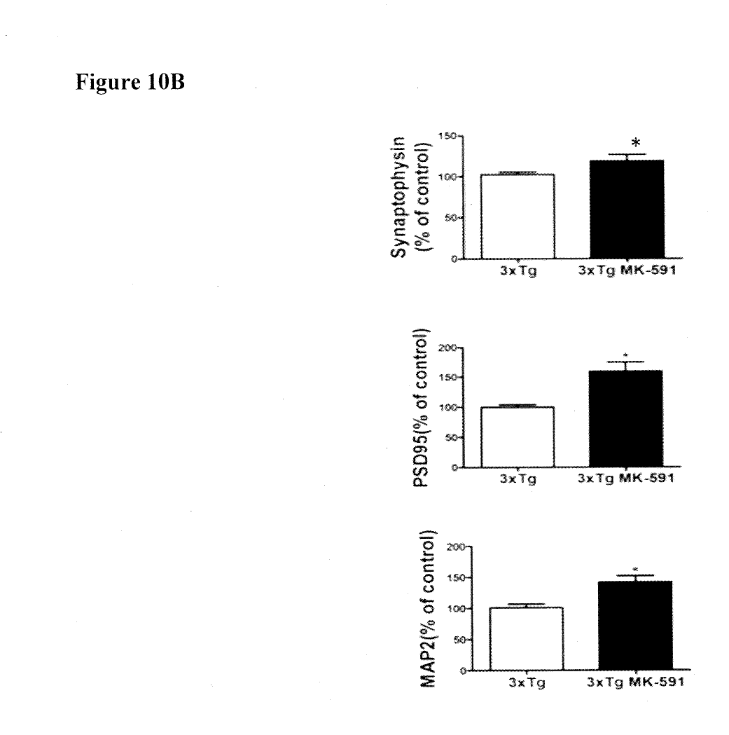

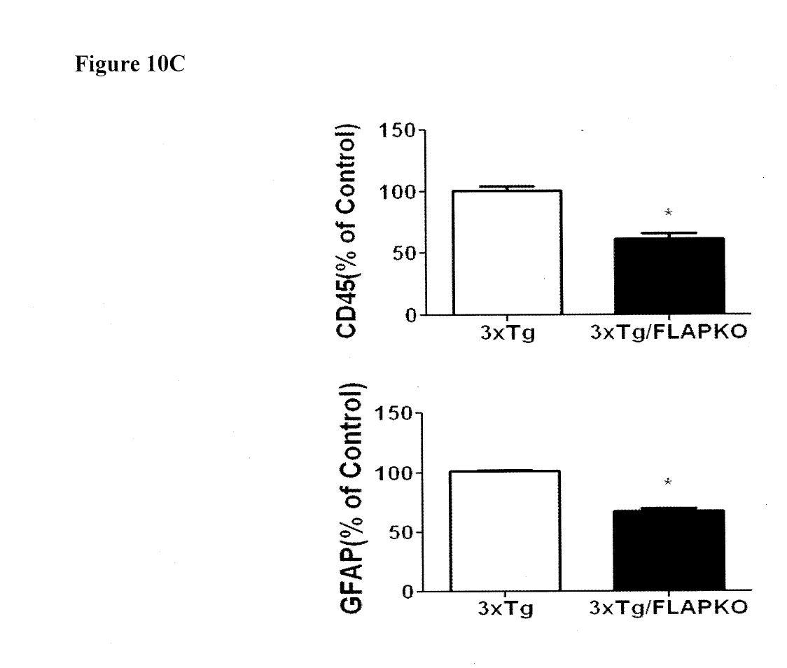

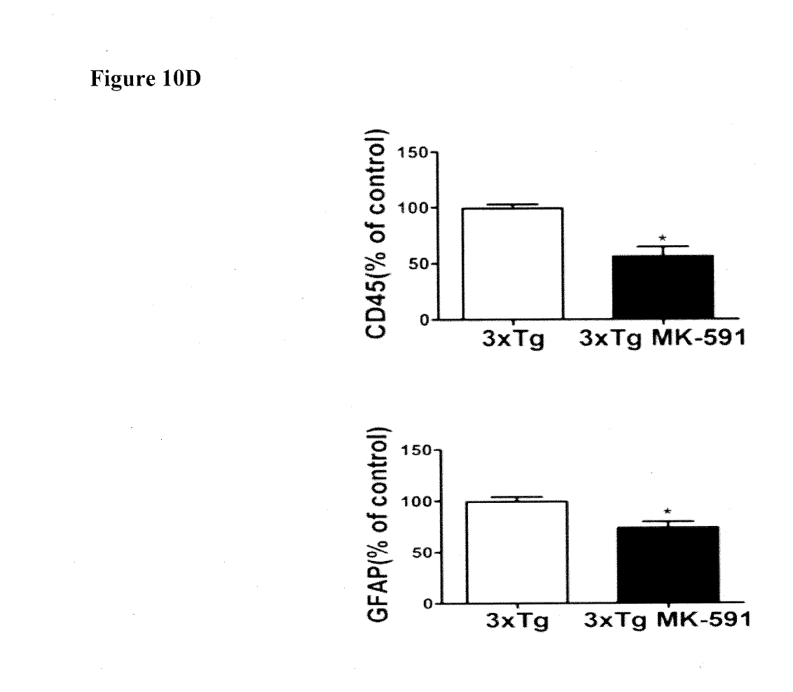

Since changes in tau phosphorylation state and solubility have been correlated with modifications of synaptic integrity in AD, we then evaluated this aspect of the 3xTg mice phenotype. Compared with the control group, 3xTg-FLAPKO and 3xTg mice treated with MK-591 had a significant increase in the steady state levels of two main synaptic proteins: post-synaptic density protein 95 (PSD-95) and Synaptophysin (SYP) (FIG. 10 A-B). A similar result was obtained also when the dendritic protein MAP2 was assayed (FIG. 10 A-B). These results were further confirmed in brain sections of the same mice when they were assessed by immunohistochemical analyses (not shown). Finally, we observed that compared with brain homogenates from 3xTg, both 3xTg-FLAPKO and MK-591 treated mice had a significant decrease in GFAP and CD45 immunoreactivities, markers of astrocytes and microglia cells activation respectively (FIG. 10 C-D).

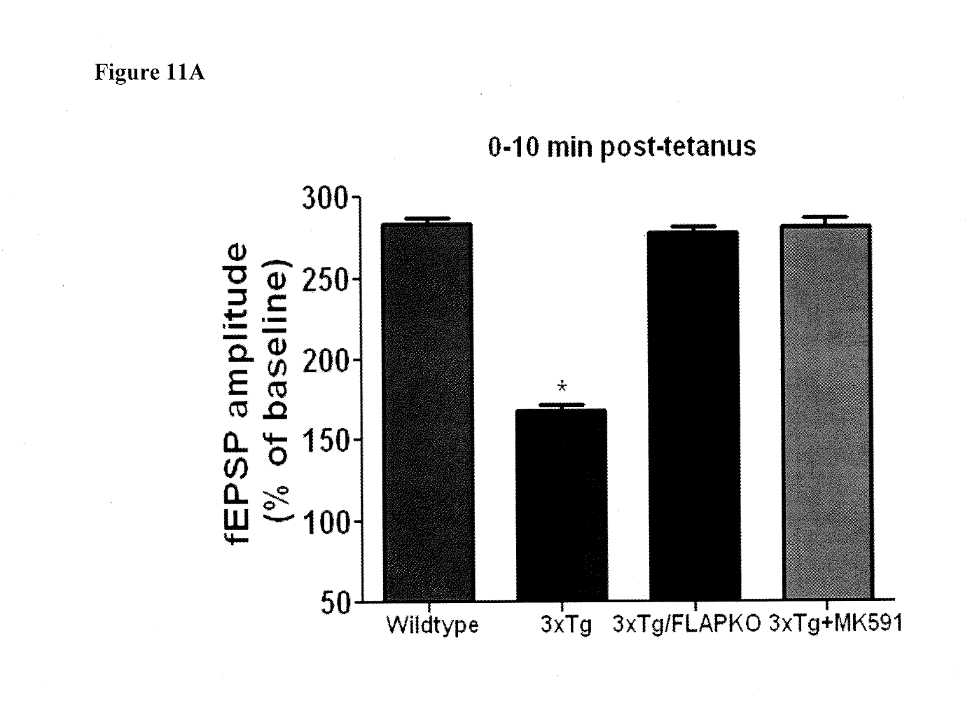

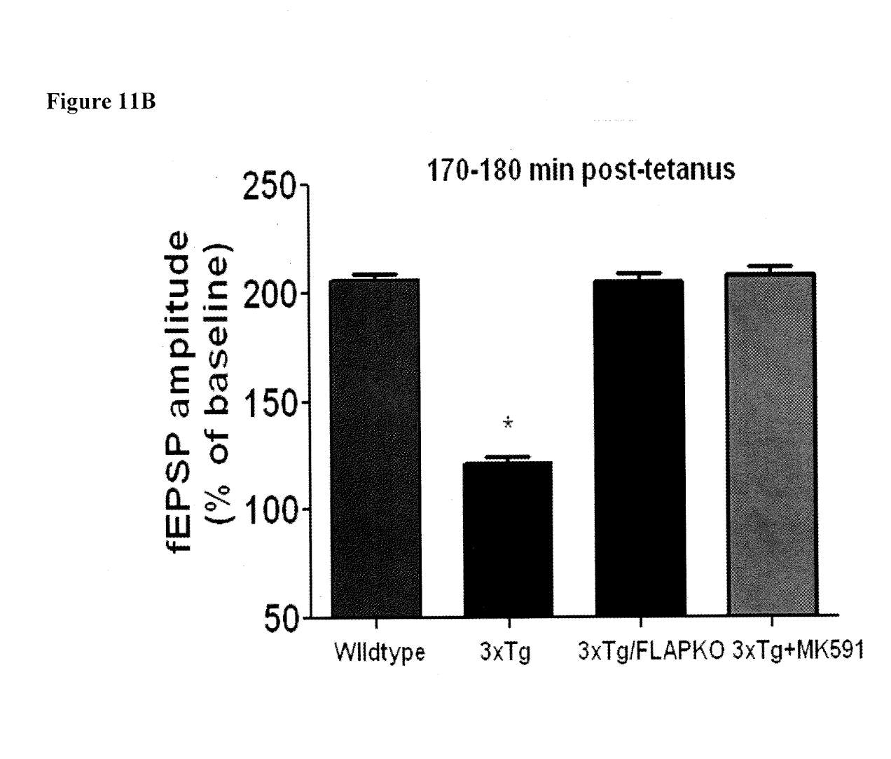

Since the genetic absence and pharmacological inhibition of FLAP in 3xTg mice yielded significant improvements in memory at an early age (6 months) prior to plaque and tangle pathology, we explored its effect on synaptic function at this young age. Initially, we investigated basal synaptic transmission by generating input/output (I/O) curves and measuring field-excitatory postsynaptic potentials (fEPSPs) elicited in the CA1 region of the hippocampus by stimulation of the Schaffer collaterals at increasing strength of stimulations and intensities. There were no differences observed in the I/O curves among the groups considered (WT, 3xTg, 3xTg-FLAPKO, 3xTg MK-591) (not shown). We then measured short-term plasticity by examining paired-pulse facilitation (PPF), which is due to an activity-dependent presynaptic modulation of transmitter release (Zucker R S et al. Annu Rev Physiol 2002, 64:355-405). Similar to what was observed in the I/O curves, no differences were noted in PPF among any of the groups analyzed (not shown). Finally, we investigated long-term potentiation (LTP) in the CAl region of the hippocampus, which is thought to measure neuronal plasticity along with being a key player in memory and cognition (Bliss TVP et al. Nature 1993, 361:31-39). In this test we found that, compared with WT, 3xTg mice had a significant reduction in LTP responses. However, the genetic absence and pharmacological inhibition of FLAP with MK-591 in the 3xTg mice completely restored the LTP responses to a level comparable to that of the WT mice (FIG. 11A-B). These results indicate that treatment with MK-591 ameliorates synaptic integrity.

The disclosures of each and every patent, patent application, and publication cited herein are hereby incorporated herein by reference in their entirety.

One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

1. A method for the treatment and/or prevention of an amyloid beta amyloidosis in a subject comprising the step of administering to the subject an effective amount of a 5-lipoxygenase-activating protein (FLAP) antagonist.

2. The method of claim 1 wherein said FLAP antagonist is MK-591, or a pharmaceutically acceptable salt thereof.

3. The method of claim 1 wherein said amount is from about 0.1 mg/day to about 10 g/day.

4. The method of claim 3 wherein said amount is from about 1 mg/day to about 5 g/day.

5. The method of claim 4 wherein said amount is from about 10 mg/day to about 1 g/day.

6. The method of claim 5 wherein said amount is from about 100 mg/day to about 800 mg/day.

7. The method of claim 3 wherein said amount is given once a day.

8. The method of claim 3 wherein said amount is divided into 2, 3 or 4 daily doses.

9. The method of claim 1 wherein said disease is Alzheimer's Disease, Cerebral amyloid angiopathy, Lewy body dementia or inclusion body myositis.

10. The method of claim 1 wherein said disease is Alzheimer's Disease.

11. The method of claim 1 wherein said subject is human.

12. A method for decreasing or preventing the deposition of beta amyloid in the brain of a subject comprising the step of administering to a subject an effective amount of a FLAP antagonist.

13. The method of claim 12 wherein said FLAP antagonist is MK-591, or a pharmaceutically acceptable salt thereof.

14. The method of claim 12 wherein said amount is from about 0.1 mg/day to about 10 g/day.

15. The method of claim 14 wherein said amount is from about 1 mg/day to about 5 g/day.

16. The method of claim 15 wherein said amount is from about 10 mg/day to about 1 g/day.

17. The method of claim 16 wherein said amount is from about 100 mg/day to about 800 mg/day.

18. The method of claim 14 wherein said amount is given once a day.

19. The method of claim 14 wherein said amount is divided into 2, 3 or 4 daily doses.

20. The method of claim 12 wherein said disease is Alzheimer's Disease, Cerebral amyloid angiopathy, Lewy body dementia or inclusion body myositis.

21. The method of claim 12 wherein said disease is Alzheimer's Disease.

22. The method of claim 12 wherein said subject is human.
